(19) United States
(12) Patent Application Publication
(10) Pub. No.: US 2005/0158838 A1
(43) Pub. Date: Jul. 21, 2005

(54) Novel enterokinase cleavage sequences

(75) Inventors: Arthur C. Ley, Newton, MA (US); Christopher Jon Luneau, Salem, MA (US); Robert C. Ladner, Ijamsville, MD (US)

Correspondence Address: FISH & RICHARDSON PC, 225 FRANKLIN ST, BOSTON, MA, 02110, US

(73) Assignee: Dyax Corp., a Delaware corporation

(21) Appl. No.: 11/030,348

(22) Filed: Jan. 6, 2005

Related U.S. Application Data
(63) Continuation of application No. 09/884,767, filed on Jun. 19, 2001.
(60) Provisional application No. 60/367,345, filed on Jun. 19, 2000.

(51) Int. Cl. C12N 9/12; C07H 21/04; C12P 21/04; C12N 1/21; C12N 15/74

(52) U.S. Cl. 435/194; 435/69.1; 435/320.1; 435/252.3; 435/325; 536/23.2

(57) ABSTRACT

Novel enterokinase cleavage sequences are provided. Also disclosed are methods for the rapid isolation of a protein of interest present in a fusion protein construct including a novel enterokinase cleavage sequence of the present invention and a ligand recognition sequence for capturing the fusion construct on a solid substrate. Preferred embodiments of the present invention show rates of cleavage up to thirty times that of the known enterokinase cleavage substrate (Asp)₄-Lys-Ile.

### GOVERNMENT FUNDING

The present invention was developed in part with funding under the National Institute of Standards Advanced Technology Program, Cooperative Agreement No. 70NANB7H3057. The government retains certain rights in this invention as a result.

### FIELD OF THE INVENTION

The present invention relates to the discovery and use of novel enterokinase recognition sequences. The present invention also relates to the construction and expression from a host cell of a fusion protein comprising a ligand recognition sequence, a novel enterokinase recognition sequence and a protein of interest. Also disclosed is a method for utilizing the ligand and enterokinase recognition sequences to isolate a highly purified protein of interest from the fusion construct by a simple one step procedure involving the incubation of enterokinase enzyme with the fusion protein immobilized on a solid support.

### BACKGROUND

The serine protease enterokinase (EK), also known as enteropeptidase, is a heterodimeric glycoprotein present in the duodenal and jejunal mucosa and is involved in the digestion of dietary proteins. Specifically, enterokinase catalyzes the conversion, in the duodenal lumen, of trypsinogen into active trypsin via the cleavage of the acidic propeptide from trypsinogen. The activation of trypsin initiates a cascade of proteolytic reactions leading to the activation of many pancreatic zymogens. (Antonowicz, Ciba Found. Symp., 70: 169-187 (1979); Kitamoto et al., Proc. Natl. Acad. Sci. USA, 91(16): 7588-7592 (1994)). EK is highly specific for the substrate sequence (Asp)₄-Lys-Ile on the trypsinogen molecule, where it acts to mediate cleavage of the Lys-Ile bond.

EK isolated from bovine duodenal mucosa exhibits a molecular weight (MW) of 150,000 and a carbohydrate content of 35%. The enzyme is comprised of a heavy chain (MW˜115,000) and a disulfide-linked light chain (MW˜35,000). (Liepnieks et al., J. Biol. Chem., 254(5): 1677-1683 (1979)). Kitamoto et al., supra, reported that the enterokinase isolated from different organisms exhibits a heavy chain molecular weight variability of from 82-140 kDa and a light chain variability of from 35-62 kDa, depending on the organism. The heavy chain functions to anchor the enzyme in the intestinal brush border membrane and the light chain is the catalytic subunit.

The cloning and functional expression of a cDNA encoding the light chain of bovine enterokinase has been reported. (LaVallie et al., J. Biol. Chem., 268(31): 23311-23317 (1993)). The cDNA sequence codes for a 235 amino acid protein that is highly homologous with a variety of mammalian serine proteases involved in digestion, coagulation and fibrinolysis. The cDNA light chain product migrates at MW 43,000 Da on SDS-PAGE, and exhibits high levels of activity in cleaving the EK-specific fluorogenic substrate Gly-(Asp)₄-Lys-beta-naphthylamide.

U.S. Pat. No. 5,665,566 to LaVallie describes the cloning and expression of the enterokinase light chain in CHO cells and Vozza et al., Biotechnology (NY), 14(1): 77-81 (1996) describe the production of rEK_{L} from an expression vector transformed in the methylotrophic yeast Pichia pastoris.

Lu et al., J. Biol. Chem., 272(50): 31293-31300 (1997) reported that, while the enterokinase light chain, either produced recombinantly or by partial reduction of purified bovine enteropeptidase, had normal activity toward small peptides with the (Asp)₄-Lys sequence, the light chain alone had dramatically reduced activity toward trypsinogen compared to the enteropeptidase holoenzyme. Therefore, the recognition of small substrates requires only the light chain, whereas efficient cleavage of trypsinogen may also depend on the presence of the heavy chain. It has been suggested that the improved ability of the light chain alone to cleave the (Asp)₄-Lys sequence in fusion proteins with greater efficiency than the holoenzyme may be due to its ability to easily access the pentapeptide depending on its location within the folded fusion protein.

Collins-Racie et al., Biotechnology, 13(9): 982-987 (1995), reported the use of the (Asp)₄-Lys pentapeptide substrate in a fusion protein as an autocatalytic substrate for the production of recombinant light chain enterokinase (rEK_{L}). Essentially, rEK_{L} cDNA was fused in frame to the C-terminus of the coding sequence for E. coli DsbA protein, which directs secretion to the E. coli periplasmic space. These two domains were joined by the (Asp)₄-Lys linker/cleavage sequence fused immediately upstream to the N-terminus of the mature rEK_{L} domain. Collins-Racie et al. recovered a soluble DsbA/rEK_{L} fusion protein from cells expressing the gene fusion construct. Following partial purification of the fusion protein, active rEK_{L} was recovered subsequent to autocatalysis of the (Asp)₄-Lys pentapeptide.

Wang et al., Biol. Chem. Hoppe Seyler, 376(11): 681-684 (1995) describe the production of enzymatically active recombinant human chymase (rHC), a proteinase present in mast cells, by a method involving proteolytic activation from a ubiquitin fusion protein containing the enterokinase cleavage site in place of the native chymase propeptide. Wang et al. transformed E. coli with an expression vector comprising the coding sequence for ubiquitin linked to the enterokinase cleavage sequence linked to the chymase gene. The fusion protein was expressed and analyzed for enterokinase-mediated activation of chymase from the refolded fusion protein. At the highest concentration of enterokinase, approximately 2.5% of the folded fusion protein was converted into enzymatically active rHC, as evidenced in comparative studies with human chymase. From these analyses, Wang et al. concluded that the use of the enterokinase cleavage site in place of the native propeptide for activation purposes, demonstrates that the presence of the native propeptide is not essential for the folding and activation of HC expressed in recombinant systems.

Light et al., Anal. Biochem., 106: 199-206 (1980) investigated the specificity of the enterokinase holoenzyme purified to homogeneity from bovine intestinal mucosa through incubation of the enzyme with various proteins of known sequence followed by an analysis of the resulting fragments on SDS-PAGE. Analysis of the resulting protein fragments indicated that either lysine or arginine can occupy the amino acid position immediately upstream (towards the amino-terminus) of the cleaved peptide bond (the P₁ position), an acidic amino acid must occur immediately upstream of this lysine or arginine (the P₂ position) and hydrolysis was increased when an acidic amino acid occurred at the 2^{nd} and 3^{rd} amino acids upstream from the cleaved peptide bond (the P₂ and P₃ positions).

Additionally, Light and Janska, Trends Biochem. Sci., 14(3) 110-112 (1989), reported studies showing that lysyl, arginyl, or the cysteinyl derivative, S-aminoethyl cysteine, could be substituted for the basic lysine residue and that aspartyl, glutamyl, or S-carboxymethyl cysteine could be substituted for the basic arginine residues. Additionally, they reported that asparagine at the 3^{rd} amino acid position upstream from the cleaved peptide bond (known as the “scissile bond”) slowed hydrolysis by enterokinase and that changes at the 4^{th} and 5^{th} upstream positions showed greater variability but also slowed the rate of hydrolysis.

Presently, while current investigations into the advantages of utilizing the highly specific (Asp)₄-Lys enterokinase recognition sequence for various chemical and biological applications are promising, these potential applications are hindered by the enzyme/substrate kinetics which act to limit specificity and rate of hydrolysis. Therefore, since enterokinase, both natural and recombinant, is readily available in commercial quantities, it would be advantageous to identify additional enterokinase cleavage sequences that exhibit an even higher specificity as well as a higher rate of hydrolysis than currently observed with the (Asp)₄-Lys pentapeptide recognition sequence.

In particular, the discovery of new peptides that are cleaved rapidly and specifically by enterokinase would find beneficial use in the field of large scale protein purification.

### SUMMARY OF THE INVENTION

Accordingly, it is an object of the present invention to identify novel enterokinase recognition sequences. Using phage display technology, a number of novel enterokinase recognition sequences have been discovered that provide a highly specific substrate for rapid cleavage by enterokinase. In addition, based on analysis of isolated sequence data, the present invention also discloses the chemical synthesis of short peptides with improved specificity and rate of cleavage at the scissile bond over the initial sequence isolates. These short peptide sequences are about 5-10 amino acids long, more preferably 5-9 amino acids long, and most preferably 5 or 6 amino acids long. The novel enterokinase recognition sequences may be incorporated as a fusion partner into a fusion protein construct, fused to a protein of interest, or included in a fusion protein display in a recombinant genetic package, lending enterokinase cleavability to the fusion protein.

Preferred enterokinase recognition sequences of the present invention exhibit not only a high binding specificity for the enterokinase enzyme but also rapid cleavage by the enzyme at a predetermined site within the cleavage recognition domain. Such sequences are useful for the rapid purification of almost any protein of interest expressed from a host cell.

The present invention also provides DNA sequences encoding an enterokinase-cleavable fusion protein comprising a novel enterokinase recognition sequence of the present invention fused to a protein of interest. Additionally, the DNA construct optionally includes a nucleotide sequence encoding a ligand recognition sequence which specifically recognizes and binds to a ligand binding partner, such as, for instance, a streptavidin binding peptide sequence for binding a streptavidin substrate, providing a means for ready capture of the enterokinase-cleavable protein of interest, which can be released by cleavage at the enterokinase recognition sequence to yield pure protein of interest.

The enterokinase recognition sequence, with or without a ligand recognition sequence fused thereto, can be located anywhere along the fusion protein so long as the chosen location is not associated with any negative properties such as impeding or destroying the biological activity of the protein of interest. In addition, the protein of interest may be present as a complete mature protein or a mutant of a protein, such as, for example, a deletion mutant or substitution mutant.

Also provided by the current invention are methods for the isolation and purification of a protein of interest present as one domain of a larger fusion protein. The protein of interest can be easily cleaved from the rest of the fusion protein, preferably by capture of the fusion protein on a solid substrate and subsequent treatment of the immobilized complex with enterokinase. In one embodiment, the fusion protein is secreted from the host cell into a culture medium. The culture medium is passed over a column which contains a ligand binding partner, such as, for instance, streptavidin or biotin, immobilized on a substrate. The ligand recognition sequence of the fusion protein forms a binding complex with the ligand binding partner thereby immobilizing or capturing the fusion protein on the column. Enterokinase is then added to the column to cleave the protein of interest from the captured fusion complex and the protein of interest is released from the fusion protein complex bound to the ligand binding partner. The purified protein of interest is collected in the flow-through supernatant.

In another embodiment, an expression vector comprising a DNA sequence encoding a fusion protein complex comprising a ligand recognition sequence, an enterokinase cleavage sequence and a protein of interest or fragment thereof may be isolated by first transfecting a host cell with the expression vector and incubating under conditions suitable for expression of the fusion protein. Most preferably, the expression vector also will include a suitable secretion signal sequence (e.g., N-terminal to the ligand recognition sequence) to effect secretion of the expression fusion protein into the culture medium.

In a batch purification process, beads coated with a ligand binding partner for the ligand recognition sequence of the fusion protein may be added directly to the culture medium containing the mature fusion protein. The beads, having captured the fusion protein, may be isolated, e.g., by filtration or immobilized in a magnetic field in the case of magnetic beads, and unwanted components of the culture medium removed. To separate the desired protein of interest from the beads and its fusion partners, enterokinase enzyme or active fragment thereof may then be added to contact the beads and incubated with the bound fusion protein. After cleavage of the fusion protein, the beads may be isolated again, and the protein of interest, now cleaved from the bead/ligand binding partner/enterokinase recognition sequence complex, may be collected in purified form.

In another embodiment, the expression vector comprising the DNA sequence encoding the fusion protein may not include a signal sequence for transport of the expressed fusion construct across the cell membrane. In this instance, the host cell may be lysed after expression of the fusion protein and the cellular debris removed from the culture medium by, for instance, filtration or centrifugation, before capture of the fusion protein on a solid substrate and subsequent treatment of the captured protein complex with enterokinase.

Specific enterokinase recognition sequences according to the present invention are shown in Tables 1-4 (infra). From analysis of cleavage data from the enterokinase recognition sequences presented herein, general formulae for two groups of preferred enterokinase sequences can be seen. Such preferred enterokinase recognitions sequences include polypeptides comprising amino acid sequences of the following general formulae:

| | |
|---|---|
| (1) | |
| Z₁-Xaa₁-Xaa₂-Xaa₃-Xaa₄-Asp-Arg- | (SEQ ID NO: 1) |
| Xaa₅-Z₂, | |

wherein Xaa₁ is an optional amino acid residue which, if present, is Ala, Asp, Glu, Phe, Gly, Ile, Asn, Ser, or Val; Xaa₂ is an optional amino acid residue which, if present, is Ala, Asp, Glu, His, Ile, Leu, Met, Gln, or Ser; Xaa₃ is an optional amino acid residue which, if present, is Asp, Glu, Phe, His, Ile, Met, Asn, Pro, Val, or Trp; Xaa₄ is Ala, Asp, Glu, or Thr; and Xaa₅ can be any amino acid residue; and wherein Z₁ and Z₂ are both optional and are, independently, polypeptides of one or more amino acids; or

| | |
|---|---|
| (2) | |
| Z₁-Xaa₁-Xaa₂-Xaa₃-Xaa₄-Glu-Arg- | (SEQ ID NO: 2) |
| Xaa₅-Z₂, | |

wherein Xaa₁ is an optional amino acid residue which, if present, is Asp or Glu; Xaa₂ is an optional amino acid residue which, if present, is Val; Xaa₃ is an optional amino acid residue which, if present, is Tyr; Xaa₄ is Asp, Glu, or Ser; and Xaa₅ can be any amino acid residue; and wherein Z₁ and Z₂ are both optional and are, independently, polypeptides of one or more amino acids.

Preferably, in both formulae (1) and (2), above, Z₁ will be a polypeptide including a ligand recognition domain or sequence useful for immobilizing the fusion protein of SEQ ID NO:1 by contact with a binding partner for said ligand, and preferably Z₂ will be a polypeptide that is or incorporates a protein of interest. Most preferably, the protein of interest will be made up of the polypeptide described by Xaa₅-Z₂, so that Xaa₅ is the N-terminus of the protein of interest, and so that enterokinase cleavage at the scissile bond Arg-Xaa₅ liberates the entire protein of interest from the enterokinase recognition sequence and Z₁ (if present). Also, preferably, Xaa₅ will be Met, Thr, Ser, Ala, Asp, Leu, Phe, Asn, Trp, Ile, Gln, Glu, His, Val, Gly, or Tyr.

An especially preferred group of enterokinase cleavage sequences includes polypeptides comprising the amino acid sequence: Asp-Ile-Asn-Asp-Asp-Arg-Xaa₅ (SEQ ID NO:3), wherein Xaa₅ can be any amino acid residue, preferably Met, Thr, Ser, Ala, Asp, Leu, Phe, Asn, Trp, Ile, Gln, Glu, His, Val, Gly, or Tyr.

Another group of preferred enterokinase cleavage sequences includes polypeptides comprising the amino acid sequence: Gly-Asn-Tyr-Thr-Asp-Arg-Xaa₅ (SEQ ID NO:4), wherein Xaa₅ can be any amino acid residue, preferably Met, Thr, Ser, Ala, Asp, Leu, Phe, Asn, Trp, Ile, Gln, Glu, His, Val, Gly, or Tyr.

In a preferred aspect of the present invention, Z₁ or Z₂ in the formulae (1) and (2) above (SEQ ID NO:1 or 2) will include a modified streptavidin ligand recognition sequence of the formula: Cys-His-Pro-Gln-Phe-Cys (SEQ ID NO:5), and preferably that sequence will be N-terminal to the enterkinase recognition sequence (i.e., will be at least a part of Z₁). Inclusion of such sequences will permit the enterokinase recognition sequence, or any polypeptide containing it, to be immobilized on a streptavidin substrate.

In addition, it is also envisioned that the phage display method of the current invention can be used to isolate additional enterokinase recognition sequences as well as optimal substrates for other enzymes of interest.

In another embodiment the present invention provides a fusion protein comprising a protein of interest fused to a ligand recognition sequence via the novel enterokinase recognition sequences of the present invention. The protein of interest can be any protein or fragment thereof capable of expression as a domain in a fusion construct. The fusion construct can be expressed as an intercellular protein in, for instance, E. coli, and isolated by disruption of the cells and removal of the fusion construct from the cellular supernatant. Alternatively, the fusion construct can include a peptide signal sequence effective for signaling secretion from the host cell producing the fusion protein. This will preclude the necessity to lyse the E. coli or other host cells to release the expressed fusion protein and thereby eliminates the need for an additional protein purification step specifically to remove unwanted cellular debris. Signal peptide sequences that are known to facilitate secretion of peptides expressed in E. coli into the culture medium include Pel B, bla, and phoA.

The ligand recognition sequence domain of the fusion construct can be any sequence which recognizes or exhibits an affinity for a binding partner such as, for instance, streptavidin. Preferred recognition sequences include the streptavidin binding sequence His-Pro-Gln-Phe (SEQ ID NO:6) and the modified streptavidin binding sequences Cys-His-Pro-Gln-Phe-Cys (SEQ ID NO:5) and Cys-His-Pro-Gln-Phe-Cys-Ser-Trp-Arg (SEQ ID NO:7). Additional preferred recognition sequences include the streptavidin binding sequences Trp-His-Pro-Gln-Phe-Ser-Ser (SEQ ID NO:210) and Pro-Cys-His-Pro-Gln-Phe-Pro-Arg-Cys-Tyr (SEQ ID NO:211). The addition of the cysteines to the modified streptavidin binding sequence makes the domain somewhat more like a protein (in that the domain obtains a 3-dimensional structure), the addition of tryptophan makes the binding sequence a better UV absorber (therefore making it easier to assay), and the addition of arginine aids solubility. In a preferred embodiment the streptavidin ligand recognition sequence or the modified streptavidin ligand recognition sequence is fused at the amino-terminal end of the novel enterokinase recognition sequences disclosed in the present application. Several such sequences can be added in tandem to provide multimeric immobilization sites.

In another embodiment, the present invention provides a DNA expression vector, for transformation of a host cell, coding for a fusion protein comprising a protein of interest fused at either the NH₂-terminus or COOH-terminus to an enterokinase recognition sequence of the present invention. The enterokinase recognition sequence may additionally be fused to a ligand recognition sequence which binds to a particular ligand and can be used to capture the ligand recognition sequence and any protein of interest attached to it, to a solid substrate. Preferably the ligand recognition sequence is positioned relative to the enterokinase recognition sequence and the protein of interest so that upon capture on a solid substrate, treatment of the fusion construct with enterokinase enzyme will release the protein of interest from the construct. Additional DNA sequences included in the expression vector may include a promoter to facilitate expression of the fusion protein in the selected host cell and preferably also a signal sequence to facilitate secretion of the fusion protein into the culture medium prior to the purification step.

In another embodiment, the expression vector does not include a signal sequence directing secretion of the expressed fusion protein into the culture medium. According to this method, after expression of the fusion protein in the host cell, the host cell is lysed and the cellular debris separated from the culture supernatant and the fusion protein by, for instance, filtration, and the protein of interest isolated according to any of the previous methods.

In accordance with the present invention, desired gene products are produced as fusion proteins expressed from host microorganisms, the fusion protein comprising a novel enterokinase cleavage sequence inserted between a ligand recognition sequence and a protein of interest. It has been found that desired peptides or proteins can be obtained in the mature form from fusion proteins produced in the above manner when the latter are treated with enterokinase capable of specifically recognizing and hydrolyzing a peptide bond within the recognition sequence. If necessary, the enterokinase may be used in combination with an aminopeptidase capable of specifically liberating a basic amino acid residue from the N-terminal side of the protein of interest or a carboxypeptidase capable of specifically liberating a basic amino acid residue from the C-terminal side of the protein of interest.

The most preferred fusion protein of the present invention, translated from an expression vector transformed in a host cell, comprises a secretion signal sequence fused to the amino-terminus of a ligand recognition sequence fused to the amino-terminus of a novel enterokinase recognition sequence of the present invention fused at its carboxy-terminus to the amino-terminal end of a protein of interest. The protein of interest may be isolated and rapidly purified in a few easy steps. Essentially, the fusion protein is expressed under suitable conditions in a host system, such as, for instance, E. Coli. After expression, the fusion protein is secreted from the host cell into the culture medium. The culture medium is then contacted with a ligand binding partner immobilized on a solid substrate under conditions suitable for binding of the ligand recognition sequence to the immobilized ligand binding partner. Treatment of the resulting complex with enterokinase releases the protein of interest from the immobilized fusion complex such that it may be subsequently isolated from the flow-through supernatant in a highly purified, biologically active form.

In another embodiment, the present invention provides a method for rapid purification of a protein of interest comprising:
(a) culturing a host cell transformed with an expression vector encoding a fusion protein comprising the elements: an enterokinase recognition sequence according to the invention, a protein of interest, and a ligand recognition sequence, the elements being expressed as a fusion construct in such a manner that each element is fully functional and no element interferes with the functionality of any other element in the construct;
(b) contacting a sample of the culture medium or cellular extract with a ligand binding partner for said ligand recognition sequence immobilized on a solid substrate;
(c) incubating the sample with enterokinase;
(d) recovering any protein of interest released from step (c).
   Optionally, one or more wash steps may be included in the purification process.

In another embodiment, the host cell may be lysed and the cellular debris separated from the fusion protein prior to isolation of the protein of interest.

Specific embodiments of the invention include the following:

A polypeptide comprising an enterokinase recognition sequence and having the formula:

| | |
|---|---|
| Z₁-Xaa₁-Xaa₂-Xaa₃-Xaa₄-Asp-Arg- | (SEQ ID NO: 1) |
| Xaa₅-Z₂, | |

wherein Xaa₁ is an optional amino acid residue which, if present, is Ala, Asp, Glu, Phe, Gly, Ile, Asn, Ser, or Val; Xaa₂ is an optional amino acid residue which, if present, is Ala, Asp, Glu, His, Ile, Leu, Met, Gln, or Ser; Xaa₃ is an optional amino acid residue which, if present, is Asp, Glu, Phe, His, Ile, Met, Asn, Pro, Val, or Trp; Xaa₄ is Ala, Asp, Glu, or Thr; and Xaa₅ can be any amino acid residue; and wherein Z₁ and Z₂ are both optional and are, independently, polypeptides of one or more amino acids. Preferably Xaa₁ is Asp, Xaa₂ is Ile, Xaa₃ is Asn, Xaa₄ is Asp, and Xaa₅ is Met, Thr, Ser, Ala, Asp, Leu, Phe, Asn, Trp, Ile, Gln, Glu, His, Val, Gly, or Tyr.

In a particular embodiment, the polypeptide Z₁ is a ligand recognition sequence, e.g., a streptavidin binding domain. Specific streptavidin binding domains may be selected from the sequences: His-Pro-Gln-Phe (SEQ ID NO:6), Cys-His-Pro-Gln-Phe-Cys (SEQ ID NO:5), Cys-His-Pro-Gln-Phe-Cys-Ser-Trp-Arg (SEQ ID NO:7), Trp-His-Pro-Gln-Phe-Ser-Ser (SEQ ID NO:210), Pro-Cys-His-Pro-Gln-Phe-Pro-Arg-Cys-Tyr (SEQ ID NO:211), and tandemly arranged combinations and repeats thereof.

In a further embodiment, the polypeptide Z₂ is a protein of interest. Preferably, the polypeptide Xaa₅-Z₂ is a protein of interest, i.e., the polypeptide of SEQ ID NO:1 is a fusion protein which, upon treatment with EK and cleavage of the scissile bond, yields an isolated protein of interest.

Other specific embodiments of the present invention include the following:

A polypeptide comprising an enterokinase recognition sequence and having the formula:

| | |
|---|---|
| Z₁-Xaa₁-Xaa₂-Xaa₃-Xaa₄-Glu-Arg- | (SEQ ID NO: 2) |
| Xaa₅-Z₂, | |

wherein Xaa₁ is an optional amino acid residue which, if present, is Asp or Glu; Xaa₂ is an optional amino acid residue which, if present, is Val; Xaa₃ is an optional amino acid residue which, if present, is Tyr; Xaa₄ is Asp, Glu, or Ser; and Xaa₅ can be any amino acid residue; and wherein Z₁ and Z₂ are both optional and are, independently, polypeptides of one or more amino acids. Preferably Xaa₅ is Met, Thr, Ser, Ala, Asp, Leu, Phe, Asn, Trp, Ile, Gln, Glu, His, Val, Gly, or Tyr.

In a particular embodiment, the polypeptide Z₁ is a ligand recognition sequence, e.g., a streptavidin binding domain. Specific streptavidin binding domains may be selected from the sequences: His-Pro-Gln-Phe (SEQ ID NO:6), Cys-His-Pro-Gln-Phe-Cys (SEQ ID NO:5), Cys-His-Pro-Gln-Phe-Cys-Ser-Trp-Arg (SEQ ID NO:7), Trp-His-Pro-Gln-Phe-Ser-Ser (SEQ ID NO:210), Pro-Cys-His-Pro-Gln-Phe-Pro-Arg-Cys-Tyr (SEQ ID NO:211), and tandemly arranged combinations and repeats thereof.

In a further embodiment, the polypeptide Z₂ is a protein of interest. Preferably, the polypeptide Xaa₅-Z₂ is a protein of interest, i.e., the polypeptide of SEQ ID NO:1 is a fusion protein which, upon treatment with EK and cleavage of the scissile bond, yields an isolated protein of interest.

Preferred enterkinase recognition sequences according to the invention may be selected from the group consisting of SEQ ID NOs: 10-73 and 75-193, as shown in Tables 1, 2, 3, and 4 (infra).

In a preferred embodiment, the invention provides a polynucleotide, encoding an enterokinase cleavable fusion protein including the following domains, arranged in the direction of amino-terminus to carboxy-terminus: a ligand recognition sequence, an enterokinase recognition sequence having the formula Asp-Ile-Asn-Asp-Asp-Arg (SEQ ID NO:208) or Gly-Asn-Tyr-Thr-Asp-Arg (SEQ ID NO:209), and a protein of interest. Vectors comprising circular DNA and including said polynucleotide are also contemplated. Expression vectors comprising the polynucleotide operably linked to a promoter sequence for expression in a recombinant host are also contemplated. Expression vectors further comprising a signal sequence operably linked to the polynucleotide, i.e., for effecting secretion of the expressed fusion protein into a culture medium are also contemplated. Recombinant prokaryotic or eukaryotic host cells transformed with such vectors also are contemplated.

Additional embodiments of the present invention include the following:

A method for isolating a protein of interest comprising:

(a) culturing a recombinant host cell expressing a recombinant polynucleotide encoding an enterokinase cleavable fusion protein including the following domains, arranged in the direction of amino-terminus to carboxy-terminus: a ligand recognition sequence, an enterokinase recognition sequence having the formula:

| | |
|---|---|
| Xaa₁-Xaa₂-Xaa₃-Xaa₄-Asp-Arg-Xaa₅, | (SEQ ID NO: 206) |

wherein Xaa₁ is an optional amino acid residue which, if present, is Ala, Asp, Glu, Phe, Gly, Ile, Asn, Ser, or Val; Xaa₂ is an optional amino acid residue which, if present, is Ala, Asp, Glu, His, Ile, Leu, Met, Gln, or Ser; Xaa₃ is an optional amino acid residue which, if present, is Asp, Glu, Phe, His, Ile, Met, Asn, Pro, Val, or Trp; Xaa₄ is Ala, Asp, Glu, or Thr; and Xaa₅ can be any amino acid residue; or

| | |
|---|---|
| Xaa₁-Xaa₂-Xaa₃-Xaa₄-Glu-Arg-Xaa₅, | (SEQ ID NO: 207) |

wherein Xaa₁ is an optional amino acid residue which, if present, is Asp or Glu; Xaa₂ is an optional amino acid residue which, if present, is Val; Xaa₃ is an optional amino acid residue which, if present, is Tyr; Xaa₄ is Asp, Glu, or Ser; and Xaa₅ can be any amino acid residue, and a protein of interest, under conditions suitable for expression of said fusion protein;
(b) contacting the expressed fusion protein with a binding ligand immobilized on a solid support under conditions suitable for formation of a binding complex between the binding ligand and the ligand recognition sequence;
(c) contacting the binding complex with enterokinase; and
(d) recovering the protein of interest.

Where said fusion protein is not secreted on expression, the foregoing method may optionally include the further steps, after step (a), of lysing the host cells and separating the cellular debris from the lysate. Where said fusion protein is secreted on expression, the foregoing method may optionally include the further step of collecting the culture media containing the secreted fusion protein.

In the foregoing method, said fusion protein preferably has the formula:

| | |
|---|---|
| Z₁-Xaa₁-Xaa₂-Xaa₃-Xaa₄-Asp-Arg- | (SEQ ID NO: 1) |
| Xaa₅-Z₂, | |

wherein Xaa₁ is an optional amino acid residue which, if present, is Ala, Asp, Glu, Phe, Gly, Ile, Asn, Ser, or Val; Xaa₂ is an optional amino acid residue which, if present, is Ala, Asp, Glu, His, Ile, Leu, Met, Gln, or Ser; Xaa₃ is an optional amino acid residue which, if present, is Asp, Glu, Phe, His, Ile, Met, Asn, Pro, Val, or Trp; Xaa₄ is Ala, Asp, Glu, or Thr; and Xaa₅ can be any amino acid residue; Z₁ is a polypeptide comprising the sequence His-Pro-Gln-Phe-Ser-Ser-Pro-Ser-Ala-Ser-Arg-Pro-Ser-Glu-Gly-Pro-Cys-His-Pro-Gln-Phe-Pro-Arg-Cys-Tyr-Ile-Glu-Asn-Leu-Asp-Glu-Phe-Ser-Gly-Leu-Thr-Asn-Ile (SEQ ID NO:84), and Xaa₅-Z₂ is a protein of interest.

In another preferred embodiment of the foregoing method, the fusion protein has the formula:

| | |
|---|---|
| Z₁-Xaa₁-Xaa₂-Xaa₃-Xaa₄-Glu-Arg- | (SEQ ID NO: 2) |
| Xaa₅-Z₂, | |

wherein Xaa₁ is an optional amino acid residue which, if present, is Asp or Glu; Xaa₂ is an optional amino acid residue which, if present, is Val; Xaa₃ is an optional amino acid residue which, if present, is Tyr; Xaa₄ is Asp, Glu, or Ser; and Xaa₅ can be any amino acid residue; Z₁ is a polypeptide comprising the sequence His-Pro-Gln-Phe-Ser-Ser-Pro-Ser-Ala-Ser-Arg-Pro-Ser-Glu-Gly-Pro-Cys-His-Pro-Gln-Phe-Pro-Arg-Cys-Tyr-Ile-Glu-Asn-Leu-Asp-Glu-Phe-Ser-Gly-Leu-Thr-Asn-Ile (SEQ ID NO:84), and Xaa-Z₅ is a protein of interest. Most preferably, Xaa₅ is Met, Thr, Ser, Ala, Asp, Leu, Phe, Asn, Trp, Ile, Gln, Glu, His, Val, Gly, or Tyr.

In a further embodiment of the present invention, a method is provided for isolating a genetic package of interest comprising the steps:
(a) expressing in a genetic package a fusion protein comprising a protein of interest fused to an enterokinase cleavage sequence fused to a polypeptide expressed on the surface of said genetic package;
(b) contacting the genetic package with a ligand for the protein of interest, which ligand is capable of being immobilized on a solid support, under conditions suitable for the formation of a binding complex between said ligand and said protein of interest;
(c) immobilizing said ligand on a solid support, either before or after said contacting step (b),
(d) contacting the immobilized binding complex formed in step (b) with enterokinase; and
(e) recovering the genetic package of interest from said solid support.

In the foregoing method, the ligand may be immobilized, for example, by biotinylating the ligand and then binding to immobilized steptavidin or avidin. Alternatively, the ligand is immobilized by binding to an immobilized antibody that binds said ligand.

The genetic package is preferably selected from the group consisting of: bacteriophage, bacteria, bacterial spores, yeast cells, yeast spores, insect cells, eukaryotic viruses, and mammalian cells. A genetic package of interest recovered in the foregoing method may be amplified in an appropriate host including but not limited to bacterial cells, insect cells, mammalian cells, and yeast. A preferred genetic package is a filamentous bacteriophage (such as M13-derived phage) and the polypeptide expressed on the surface of said host, i.e., which anchors the fusion protein to the surface of the genetic package, is selected from the group consisting of: gene III protein (SEQ ID NO:213); domain 2::domain 3::transmembrane domain::intracellular domain of gene III protein (SEQ ID NOs:215); and domain 3::transmembrane domain::intracellular anchor of gene III protein (SEQ ID NOs:217).

In preferred embodiments, the protein of interest is an antibody or fragment thereof.

The present invention further provides a method for controlling the activity of a protein of interest comprising the steps:
(a) expressing in a recombinant host a fusion protein comprising the elements:
   (i) a first protein fused to (ii) an enterokinase cleavage sequence fused to (iii) a second protein, wherein said fusion protein has suppressed activity due to the conformation of elements (i), (ii) and (iii);
(b) treating the fusion protein with enterokinase such that said first protein and second protein are separated and at least one of said first protein and said second protein thereby exhibits the activity of a protein of interest.
   In one embodiment of the foregoing method, said second protein is the protein of interest and is a protease, and said first protein is an inhibitor of the protease. In another embodiment, said first protein is the protein of interest and is a protease, and said second protein is an inhibitor of the protease. In another embodiment, said first protein is the variable light (V_{L}) domain of an scFv antibody, and said second protein is the variable heavy (V_{H}) domain of an scFv antibody, and wherein said protein of interest is the scFv formed by the association of said first protein with said second protein. In another embodiment, said second protein is the variable light (V_{L}) domain of an scFv antibody, and said first protein is the variable heavy (V_{H}) domain of an scFv antibody, and said protein of interest is the scFv formed by the association of said first protein with said second protein.

The present invention additionally provides a method for detecting the expression of a fusion protein on the surface of a recombinant host comprising the steps:
(a) expressing, in a recombinant host, a fusion protein comprising a first protein fused to an enterokinase cleavage sequence fused to a second protein fused to a polypeptide expressed on the surface of said host;
(b) contacting the host with a ligand for said first protein immobilized on a solid support under conditions suitable for forming a binding complex between the ligand and the first protein;
(c) removing unbound materials;
(d) treating any bound complex with enterokinase;
(e) recovering hosts released from said solid support, wherein said recovered hosts are verified expressors of said fusion protein.
   In preferred embodiments, the first protein is a streptavidin-binding polypeptide and said ligand is streptavidin, and the second protein is an antibody or an antibody fragment.

The present invention also provides a method of selecting display polypeptides from a display library that have specific affinity for a target, comprising the steps:
(a) providing a display library of polypeptides comprising a multiplicity of genetic packages, wherein each genetic package expresses a fusion protein that comprises an enterokinase recognition sequence between a diplay polypeptide library member and a polypeptide that anchors the fusion protein to the genetic package,
(b) contacting the display library with a target,
(c) immobilizing the target on a solid support, either before or after said contacting step (b),
(d) separating non-binding genetic packages from bound genetic packages,
(e) treating the bound genetic packages with enterokinase, and
(f) recovering and amplifying the genetic packages released.
   Preferably, the genetic package is an M13 phage. More preferably, polypeptide that anchors the fusion protein to the genetic package comprises at least the domain 3::transmembrane domain::intracellular domain portion of the gene III protein. In particular embodiments, the display polypeptides exhibited by the genetic packages of the display library comprise human Fabs. In other embodiments, the display polypeptides comprise peptides of, e.g., ten to twenty-one amino acids in length. Specific embodiments include display peptides containing two cysteine residues.

### BRIEF DESCRIPTION OF THE DRAWINGS

FIG. 1 and FIG. 2 show the time course of enterokinase cleavage of phage isolates from five rounds of screening a substrate phage library. The tested isolates were those having recurring sequences among 90 sequenced isolates. The isolates are tested in comparison with an isolate (5-H11) containing the known enterokinase cleavage sequence DDDDK and an unselected phage displaying a polypeptide not recognized by enterokinase.

FIG. 1 shows enterokinase cleavage using 30 nM recombinant light chain enterokinase (Novagen);

FIG. 2 shows enterokinase cleavage using 130 nM recombinant light chain enterokinase.

### DEFINITIONS

As used herein, the term “recombinant” is used to describe non-naturally altered or manipulated nucleic acids, host cells transfected with exogenous nucleic acids, or polypeptides expressed non-naturally, through manipulation of isolated DNA and transformation of host cells. Recombinant is a term that specifically encompases DNA molecules which have been constructed in vitro using genetic engineering techniques, and use of the term “recombinant” as an adjective to describe a molecule, construct, vector, cell, polypeptide or polynucleotide specifically excludes naturally occurring such molecules, constructs, vectors, cells, polypeptides or polynucleotides.

The term “bacteriophage”, as used herein, is defined as a bacterial virus containing a DNA core and a protective shell built up by the aggregation of a number of different protein molecules. The term “Ff phage”, as used herein, denotes phage selected from the set comprising M13, f1, and fd and their recombinant derivatives. The term “filamentous phage”, as used herein denotes the phage selected from the set comprising Ff phage, IKe, Pf1, Pf3, and other related phage known in the art. Bacteriophage include filamentous phage, phage lambda, Ti, T7, T4, and the like. The terms “bacteriophage” and “phage” are used herein interchangeably. Unless otherwise noted, the terms “bacteriophage” and “phage” also encompass “phagemids”, i.e., plasmids which contain the packaging signals of filamentous phage such that infectious phage-like particles containing the phagemid genome can be produced by coinfection of the host cells with a helper phage. A particularly useful phage for the isolation of enterokinase cleavage sequences of the invention via phage display technology is the recombinant, single-stranded DNA, filamentous M13 phage and its derivatives. In the present application, reference to “an M13 phage” encompasses both M13 phage (wild-type) and phage derived from M13 phage (i.e., “M13-derived phage”). Such M13-derived phage contain DNA that encodes all the polypeptides of wild type M13 phage and which can infect F+E. coli to produce infectious phage particles. M13-derived phage, in other words, include functional versions of all of the wild-type M13 genes. The native M 13 genes may have been altered in M 13-derived phage, for various purposes familiar to those in the art, e.g., incorporation of silent mutations, truncations of native genes that do not affect viability or infectivity of the phage, removal or insertion of restriction sites, or addition of non-native genes into intergenic regions of the M13 genome. The term “an M13 phage” specifically includes such phage as M13mp18, M13mp7, M13mp8, M13mp9. See, U.S. Pat. No. 5,233,409; U.S. Pat. No. 5,403,484; U.S. Pat. No. 5,571,698, all incorporated herein by reference.

The term “genetic package”, as used herein, denotes a package that contains a genetic message encoding at least one protein that, in suitable circumstances, assembles into the package and is at least partly exposed on the package surface. Genetic packages include bacteriophages, bacterial cells, spores, eukaryotic viruses, and eukaryotic cells.

The term “host”, as used herein, denotes a cell type in which genetic packages can be grown. Hosts include bacterial cells, insect cells, mammalian cells, and yeast. Some genetic packages are their own hosts, such as yeast and bacterial cells. For viral genetic packages, a separate host cell is required. Suitable hosts for filamentous phage are gram negative bacteria, such as E. coli. A suitable host for baculovirus is insect cells (see, Ojala, et al., Biochem. Biophys. Res. Commun., 284(3): 777-84 (2001)).

The term “enterokinase” as used herein is a pancreatic hydrolase which facilitates the cleavage and activation of trypsinogen into trypsin as part of the catalytic cascade involved in the digestive process. “Enterokinase” includes both the native enzyme isolated from any source as well as the enzyme produced by recombinant techniques. The enterokinase described herein may exist as a dimer comprising a disulfide-linked heavy chain of approximately 120 kDa and a light chain of approximately 47 kDa. Alternatively, the light chain alone, which contains the catalytic domain, may be used. The light chain may be isolated from a native source or produced recombinantly.

The term “enterokinase recognition sequence” as used herein, denotes those sequences, usually a short polypeptide of fewer than 30 amino acids, which are contacted and cleaved by the enterokinase enzyme. The terms “enterokinase recognition sequence” and “enterokinase cleavage sequence” are used herein interchangeably.

The term “enterokinase recognition domain” as used herein, denotes the complete sequence of amino acids which must be present in order for the enterokinase enzyme to recognize and cleave a specific site within the “enterokinase recognition domain”, regardless of whether those sequences come in direct physical contact with the enzyme or are in close proximity to the actual site of cleavage.

The term “scissile bond” as used herein, denotes the specific peptide bond joining consecutive amino acids via an amide linkage that is cleaved by the enterokinase enzyme. By standard nomenclature, the scissile bond occurs between the P₁ and P₁′ amino acids within the enterokinase recognition sequence.

The term “ligand recognition sequence” as used herein, denotes a sequence of amino acids recognizing, that is, binding to, a known ligand or binding partner. If utilized in the process of isolating and purifying a protein or protein fragment, it is desirable for the ligand recognition sequence to exhibit a high specificity and high affinity for the ligand or binding partner. Examples of a ligand recognition sequence would include streptavidin (or avidin), which would recognize a biotin binding partner, or a streptavidin binding sequence (see, e.g., SEQ ID NO:5), which would form a binding complex with a streptavidin binding partner. Other examples of ligand binding partners include antibodies raised against a specific peptide antigen, which peptide antigen would be suitable for use as a ligand recognition sequence. Other examples of specific ligand recognition sequences include the Myc-tag (Munro & Pelham, Cell, 46: 291-300 (1986); Ward et al., Nature, 341: 544-546 (1989), the Flag peptide (Hopp et al., BioTechnology, 6: 1204-1210 (1988), the KT3 epitope peptide (Martin et al., Cell, 63: 843-849 (1990); Martin et al., Science, 255: 192-194 (1992), an α-tubulin epitope peptide (Skinner et al., J. Biol. Chem., 266: 14163-14166 (1991), polyhistidine tags (esp. hexahistidine tails), chitin binding domain (CBD), maltose binding protein (MBP), and the T7 gene 10-protein peptide tag (Lutz-Freyermuth et al., Proc. Natl. Acad. Sci. USA, 87: 6393-6397 (1990), all of which have been used successfully for the detection and in some cases also for the purification of a recombinant gene product.

The term “fusion protein” as used herein, denotes a polypeptide formed by expression of a hybrid gene made by combining more than one gene sequence. Typically a fusion protein is produced by cloning a cDNA into an expression vector in-frame with an existing gene.

The term “protein of interest” as used herein, denotes any protein, fragment thereof, or polypeptide of any length which may be isolated and purified from its native source, or produced by recombinant DNA techniques and expressed from its native source or from a recombinant host cell, or produced by any chemical synthesis method.

The term “display library”, as used herein, denotes a plurality of genetic packages that differ only in the protein or peptide displayed. The displayed protein or peptide can be highly homologous in parts and variable in other parts, such as in a display library of Fabs. A library of displayed peptides may show no internal homology other than length and common flanking sequences or might have fixed internal amino acids, such as cysteines. A display library may also comprise a collection of cDNAs from a given cell type all fused to the same anchor protein and displayed on the same genetic package.

### DETAILED DESCRIPTION

The present invention provides novel, highly specific and rapidly cleaved enterokinase recognition sequences. The novel enterokinase recognition sequences of the present invention are small polypeptides of three or more residues which provide a substrate specifically recognized and cleaved by recombinant light chain enterokinase.

The present invention also contemplates a DNA sequence encoding an enterokinase cleavage sequence according to the present invention, preferably as part of an expression vector for transformation of a host cell and expression of a protein of interest. The expression vector preferably includes a DNA sequence that encodes a fusion protein, the fusion protein comprising several domains including, preferably, a signal sequence, a ligand recognition sequence, a novel enterokinase cleavage sequence and a protein of interest. Optionally, a fusion protein lacking a signal sequence is also envisioned by the present application.

Using standard recombinant DNA techniques, a host cell is transformed with the expression vector and under appropriate conditions, the fusion protein is expressed by the host cell. The signal sequence is desirable to facilitate secretion of the protein of interest into the culture medium prior to isolation and purification of the protein of interest. This avoids the potential problem of degradation of the protein of interest in the host cell and avoids the requirement for lysis of the host cell in turn resulting in contamination of the cell medium with unwanted proteins and other cellular debris present in a whole cell lysate. By this method, the protein of interest may be purified directly from the culture medium without the necessity of additional purification steps to remove unwanted products. However, purification of a non-secreted protein after cell lysis is also envisioned by the methods of the present invention. For instance, a protein of interest lacking a signal sequence may be purified from a fusion construct that includes a novel enterokinase cleavage sequence according to the present invention by methods described herein.

The present invention also describes construction of a cassette for expression and rapid purification of a protein of interest. Using the described cassette, virtually any protein of interest can be fused either at its NH₂-terminal or COOH-terminal end to the novel enterokinase cleavage sequences of the current invention. A purified protein of interest is easily obtained as seen by the examples described below.

As previously described, the present invention may be used to isolate and purify any number of proteins of interest. By knowing every amino acid which may occur at the P₁′ position of the enterokinase recognition domain, it can be determined if the first amino acid (occurring at either the NH₂-terminal or COOH-terminal end) of a protein of interest may be fused in a construct to the P₁ amino acid. If this first amino acid of the protein to be purified is allowed at the P₁′ position, treatment with enterokinase to remove the Pₙ-P₁ amino acids allows for the immediate isolation of a purified protein directly from the purification eluate. As used herein Pₙ-P₁ designates those amino acids which are part of the enterokinase recognition domain and occur to the amino-terminal side of the protein of interest. However, even if the first amino acid of the protein of interest must be fused “downstream” of the P₁′ position, i.e., P₂′, P₃′ etc., a highly purified protein may still be isolated from the purification eluate and the only subsequent purification step necessary is the removal of any undesired terminal amino acids from the purified protein. In many cases the extra amino acid(s) can remain attached to the protein of interest with no effect on biological activity, hence a subsequent purification/cleavage step is unnecessary.

The novel enterokinase recognition sequences of the present invention may also be used for release of a protein of interest, including without limitation an antibody or fragment thereof, that is expressed as a display on the surface of a genetic package. Following expression and display of a fusion construct that includes a surface protein or portion (stump) of a surface protein, linked to an enterokinase recognition sequence, linked to the protein of interest on the surface of the genetic package, treatment of the culture containing the genetic package or of purified genetic package with enterokinase will release the protein of interest from the fusion protein construct. According to this method, the fusion protein display on the genetic package comprises the protein of interest fused at its N-terminus or C-terminus (preferably the N-terminus) of an enterokinase recognition sequence of the present invention, and the other end (preferably the C-terminus) of the enterokinase recognition sequence is fused to a protein or portion thereof expressed on the surface of the genetic package. The host cell for display of the fusion may be any suitable cell, including without limitation bacterial cells, yeast cells, bacterial spores, or yeast spores, insect cells, or mammalian cells.

Following incubation with enterokinase, the released genetic package of interest may be collected and amplified using methods well known in the art. For example, F+ E coli cells can be infected with Ff phage so released.

In a preferred embodiment, a phage host will display a fusion protein including a protein of interest such as an antibody or a functional fragment thereof (e.g., Fab fragment, scFv, Fv, etc.) fused to an enterokinase recognition sequence of the invention, fused to a phage surface protein or portion thereof. Most preferably the fusion protein is expressed in an M13 phage. The phage surface protein used may be, e.g., the complete gene III protein of M13 filamentous bacteriophage (SEQ ID NO:213); domain 2, domain 3, the transmembrane domain, and the intracellular anchor domaim of gene III protein (SEQ ID NOs:215); domain 3 of gene III, the transmembrane domain, and the intracellular anchor domain of protein (SEQ ID NOs:217), mature gene VIII protein of a filamentous bacteriophage, or any varied, modified, truncated, or mutated form of these proteins which may be stably expressed on the surface of a host bacteriophage, preferably an M13 phage.

After expression and display on the surface of the bacteriophage, instead of releasing the protein of interest by incubating the bacteriophage with enterokinase, the protein of interest may be isolated by binding the expressed fusion protein with a ligand for the protein of interest, e.g., an antigen in the case of an antibody or antibody fragment of interest. The ligand may be immobilized on a column or other solid support or suspended in a liquid medium. After removal of unbound material by washing the support or filtering of the culture medium etc., the ligand/phage display complex is incubated with enterokinase to release the genetic package, and the genetic package of interest (carrying the gene encoding the displayed protein of interest) may be thereafter collected by elution from the ligand. The recovered genetic packages can then be amplified in suitable hosts. The enterokinase cleavage sequences disclosed herein may also be utilized as a cleavable linker to an inhibitor polypeptide, to control the activity, specificity, half-life or other function of a particular protein of interest. For instance, a fusion protein comprising, for example, a protease fused to one terminus of a novel enterokinase cleavage sequence, and an inhibitor for the protease fused to the other terminus of the enterokinase cleavage sequence, may be expressed from a host cell or displayed on the surface of a host cell or phage, such that the protease is inactive in the presence of the inhibitor. When activation or removal of the influence of the inhibitor is desired, incubation of the fusion protein with enterokinase dissociates the inhibitor from the protease, thereby liberating the protease of the inhibitor.

In a similar type of fusion construct, an enterokinase recognition sequence according to the invention may be used as a linking sequence between the light chain and heavy chain elements of a single chain antibody or scFv fragment that is expressed in a recombinant host cell or displayed on a display host such as a genetic package. Incubation of the fusion with enterokinase will eliminate the linkage between the heavy and light chain elements, permitting the heavy and light chain elements (e.g., V_{H} and V_{L} domains in the case of a scFv) to associate more freely, i.e., without any steric constraint from the linker.

The enterokinase recognition sequences disclosed herein may also be used to confirm the proper expression and/or display of a fusion protein on the surface of a host cell or bacteriophage. In this embodiment the fusion protein display comprises a protein of interest, fused to an enterokinase recognition sequence, fused to a ligand marker, for example, a streptavidin-binding peptide. After expression and display on the surface of the host cell or bacteriophage, the construct is contacted with streptavidin (Sv) immobilized on a column or other support. Hosts properly displaying the fusion will bind to immobilized ligand (e.g., Sv) while non-displaying hosts can be washed away. Incubation with enterokinase allows isolation of the bound hosts. These display-verified hosts may then be used in selections to identify proteins of interest that bind to targets of interest, e.g., by re-culturing the recovered display-verified binders and pre-treating them with enterokinase, leaving an unencumbered protein of interest display.

The enterokinase recognition sequences of the present invention can be used in selecting proteins or peptides displayed on genetic packages. The display library is prepared with an enterokinase recognition sequence positioned between the displayed library members and the anchor domain of the display fusion protein. The library of genetic packages are brought into contact with a target protein. The target protein is immobilized either before or after it is allowed to bind members of the display library. Non-binding members of the library are washed away. The immobilized genetic packages are treated with enterokinase and packages that are released are cultured. For example, Ff packages are used to infect E. coli, while display yeast genetic packages are grown in suitable growth medium. The advantage of this method is that buffer conditions need not be changed and the released packages are highly likely to have been bound by way of the displayed protein or peptide rather than some non-specific interaction with the body of the genetic package.

Identification of Novel Enterokinase Recognition Sequences

To identify novel enterokinase cleavage sequences, a substrate phage library, having a diversity of about 2×10⁸ amino acid sequences, was screened against enterokinase. The substrate phage library was designed to include a peptide-variegated region in the display polypeptide. This region consisted of 13 consecutive amino acids, and the display polypeptide design allowed any amino acid residue except cysteine to occur at each position. The substrate phage library also was characterized by inclusion of an N-terminal tandem arrangement of a linear and a disulfide-constrained streptavidin recognition sequence. The screen was carried through a total of 5 rounds of increasing stringency to obtain phage that could be released by incubation with recombinant light chain enterokinase (obtained from Novagen, Madison, Wis.) after binding to immobilized streptavidin. 90 isolates remaining after the 5^{th} round of screening were randomly chosen for further sequence analysis.

DNA sequence analysis of the 90 round 5 isolates demonstrated a substantial sequence collapse. When the isolates were grouped by sequence similarity, 82 of the 90 isolates contained one or more examples (for a total of 99 occurrences) of a simple dipeptide motif consisting of an acidic residue (Asp or Glu) followed on the carboxyl side by a basic residue. The observed frequencies of the dipeptides among the 99 instances were: Asp-Arg (DR) 66%, Asp-Lys (DK) 18%, Glu-Arg (ER) 14%, and Glu-Lys (EK) 4%.

Sequences that occurred multiple times were examined further in comparison to an isolate containing the known EK cleavage sequence (Asp)₄-Lys and an unselected (irrelevant) control. Of these isolates, several were found that cleaved more rapidly than a test sequence containing (Asp)₄-Lys (see Examples, infra).

Preparation of Phase Display Library

The enterokinase recognition sequences of the present invention were isolated from a diverse library of potential enterokinase recognition sequences fused to streptavidin recognition sequences displayed on the surface of bacteriophage. A phage display library with a display sequence diversity of 10⁸ or more may be constructed according to the methods disclosed, for example, in Kay et al., Phage Display of Peptides and Proteins: A Laboratory Manual (Academic Press, Inc., San Diego 1996) and U.S. Pat. No. 5,223,409 (Ladner et al.), and Dower et al., U.S. Pat. No. 5,432,018, incorporated herein by reference. An oligonucleotide library is inserted in an appropriate vector encoding a bacteriophage structural protein, preferably an accessible phage protein, such as a bacteriophage coat protein. Although a variety of bacteriophage may be employed in the present invention, the vector is, or is derived from, a filamentous bacteriophage, such as, for example, f1, fd, Pf1, M13, etc.

The phage vector is chosen to contain or is constructed to contain a cloning site located in the 5′ region of the gene encoding the bacteriophage structural protein, so that the enterokinase recognition sequence is accessible to the enzyme in the process of identifying novel enterokinase recognition sequences.

An appropriate vector allows oriented cloning of the oligonucleotide sequences encoding the recognition sequences of the present invention so that the recognition sequence is expressed close to the N-terminus of the mature coat protein. The coat protein is typically expressed as a preprotein, having a leader sequence. Thus, it is preferred that the oligonucleotide library is inserted so that the N-terminus of the processed bacteriophage outer protein is the first residue of the peptide, i.e., between the 3′-terminus of the sequence encoding the leader protein and the 5′-terminus of the sequence encoding the mature protein or a portion of the 5′-terminus.

The library is constructed by cloning an oligonucleotide which contains the potential enterokinase recognition sequence (and a streptavidin or other ligand recognition sequence) into the selected cloning site. Using known recombinant DNA techniques (see generally, Sambrook et al., Molecular Cloning, A Laboratory Manual, 2d ed., Cold Spring Harbor Laboratory Press, Cold Spring Harbor, N.Y., (1989), incorporated herein by reference), an oligonucleotide may be constructed which, inter alia, removes unwanted restriction sites and adds desired ones, reconstructs the correct portions of any sequences which have been removed, inserts the spacer, conserved or framework residues, if any, and corrects the translation frame (if necessary) to produce active, infective phage. The central portion of the oligonucleotide will generally contain one or more recognition sequences and any additional residues such as, for example, any spacer or framework residues. The sequences are ultimately expressed as peptides (with or without spacer or framework residues) fused to or in the N-terminus of the mature coat protein on the outer, accessible surface of the assembled bacteriophage particles.

The variable enterokinase recognition sequences of the oligonucleotide comprise the source of the library. The size of the library will vary according to the number of variable codons, and hence the size of the peptides, which are desired. Generally the library will be at least about 10⁶ members, usually at least 10⁷ and typically 10⁸ or more members.

To generate the collection of oligonucleotides which forms a series of codons encoding a random collection of possible enterokinase recognition sequences and which is ultimately cloned into the vector, a codon motif is used, such as (NNK)ₓ, where N may be A, C, G, or T (nominally equimolar), K is G or T (nominally equimolar), and x is typically up to about 5, 6, 7, or 8 or more, thereby producing libraries of penta-, hexa-, hepta-, and octa-peptides or more. The third position may also be G or C, designated “S”. Thus, NNK or NNS (i) code for all the amino acids, (ii) code for only one stop codon, and (iii) reduce the range of codon bias from 6:1 to 3:1. It should be understood that with longer peptides, the size of the library which is generated may become a constraint in the cloning process. The expression of peptides from randomly generated mixtures of oligonucleotides in appropriate recombinant vectors is discussed in Oliphant et al., Gene 44: 177-183 (1986), incorporated herein by reference.

An exemplified codon motif, (NNK)₆, produces 32 codons, one for each of 12 amino acids, two for each of five amino acids, three for each of three amino acids and one (amber) stop codon. Although this motif produces a codon distribution as equitable as available with standard methods of oligonucleotide synthesis, it results in a bias against peptides containing one-codon residues. For example, a complete collection of hexacodons contains one sequence encoding each peptide made up of only one-codon amino acids, but contains 729 (3⁶) sequences encoding each peptide with only three-codon amino acids.

An alternative approach to minimize the bias against one-codon residues involves the synthesis of 20 activated tri-nucleotides, each representing the codon for one of the 20 genetically encoded amino acids. These are synthesized by conventional means, removed from the support but maintaining the base and 5′-OH-protecting groups, and activated by the addition of 3′ O-phosphoramidite (and phosphate protection with beta cyanoethyl groups) by the method used for the activation of mononucleosides, as generally described in McBride and Caruthers, Tetrahedron Letters 22: 245 (1983), which is incorporated herein by reference. Degenerate “oligocodons” are prepared using these trimers as building blocks. The trimers are mixed at the desired molar ratios and installed in the synthesizer. The ratios will usually be approximately equimolar, but may be a controlled unequal ratio to obtain the over- to under-representation of certain amino acids coded for by the degenerate oligonucleotide collection. The condensation of the trimers to form the oligocodons is done essentially as described for conventional synthesis employing activated mononucleosides as building blocks. See generally, Atkinson and Smith, Oligonucleotide Synthesis, M. J. Gain, ed. p. 35-82 (1984) incorporated herein by reference. Thus, this procedure generates a population of oligonucleotides for cloning that is capable of encoding an equal distribution (or a controlled unequal distribution) of the possible peptide sequences. This approach may be especially useful in generating longer peptide sequences, since the range of bias produced by the (NNK)₆ motif increases by three-fold with each additional amino acid residue.

When the codon motif is (NNK)ₙ, as defined above, and when n equals 8, there are 2.6×10¹⁰ possible octapeptides. A library containing most of the octapeptides may be difficult to produce. Thus, a sampling of the octapeptides may be accomplished by constructing a subset library using from about 0.1%, and up to as much as 1%, 5%, or 10% of the possible sequences, which subset of recombinant bacteriophage particles is then screened. As the library size increases, smaller percentages are acceptable. If desired, to extend the diversity of a subset library, the recovered phage subset may be subjected to mutagenesis and then subjected to subsequent rounds of screening. This mutagenesis step may be accomplished in two general ways: the variable region of the recovered phage may be mutagenized, or additional variable amino acids may be added to the regions adjoining the initial variable sequences according to methods well known in the art.

A variety of techniques can be used in the present invention to diversify a peptide library or to diversify around peptides found in early rounds of screening to have sufficient cleavability. In one approach, the positive phage (those identified in an early round of screening) are sequenced to determine the identity of the active peptides. Oligonucleotides are then synthesized based on these peptide sequences, employing a low level of all bases incorporated at each step to produce slight variations of the primary oligonucleotide sequences. This mixture of (slightly) degenerate oligonucleotides is then cloned into the affinity phage. This method produces systematic, controlled variations of the starting peptide sequences. It requires, however, that individual positive phage be sequenced before mutagenesis, and thus is useful for expanding the diversity of small numbers of recovered phage.

Another technique for diversifying around the recognition sequence of the selected phage-peptide involves the subtle misincorporation of nucleotide changes in the peptide through the use of the polymerase chain reaction (PCR) under low fidelity conditions. The protocol of Leund et al., Technique 1: 11-15 (1989), incorporated herein by reference, alters the ratios of nucleotides and the addition of manganese ions to produce a 2% mutation frequency.

Yet another approach for diversifying the selected phage involves the mutagenesis of a pool, or subset, of recovered phage. Phage recovered from screening are pooled and single stranded DNA is isolated. The DNA is mutagenized by treatment with, e.g., nitrous acid, formic acid, or hydrazine. These treatments produce a variety of damage in the DNA. The damaged DNA is then copied with reverse transcriptase which misincorporates bases when it encounters a site of damage. The segment containing the sequence encoding the variable peptide is then isolated by cutting with restriction nuclease(s) specific for sites flanking the variable region. This mutagenized segment is then recloned into undamaged vector DNA. The DNA is transformed into cells and a secondary library is constructed. The general mutagenesis method is described in detail in Myers et al., Nucl. Acids Res., 13: 3131-3145 (1985), Myers et al., Science, 229: 242-246 (1985), and Myers, Current Protocols in Molecular Biology, Vol. 1, 8.3.1-8.3.6, Ausebel et al., eds. (J. Wiley and Sons, New York, 1989), each of which is incorporated herein by reference.

In the second general approach, that of adding additional amino acids to a peptide or peptides found to be cleavable, a variety of methods are available. In one, the sequences of peptides selected in early screening are determined individually and new oligonucleotides, incorporating the determined sequence and an adjoining degenerate sequence, are synthesized. These are then cloned to produce a secondary library.

In another approach which adds a second variable sequence region to a pool of peptide-bearing phage, a restriction site is installed next to the primary variable region. Preferably, the enzyme should cut outside of its recognition sequence, such as BspMI which cuts leaving a four base 5′ overhang, four bases to the 3′ side of the recognition site. Thus, the recognition site may be placed four bases from the primary degenerate region. To insert a second variable region, the pool of phage DNA is digested and blunt-ended by filling in the overhang with Klenow fragment. Double-stranded, blunt-ended, degenerately synthesized oligonucleotides are then ligated into this site to produce a second variable region juxtaposed to the primary variable region. This secondary library is then amplified and screened as before.

The peptide libraries, as described herein, have been used to identify novel amino acid sequences that may be recognized and cleaved by the enzyme enterokinase. This procedure may also be employed to identify the site-specificity of other protein modifying enzymes. By way of example, as described in Dower supra, factor Xₐ cleaves after the sequence Ile-Glu-Gly-Arg. A library of variable region codons may be constructed, for example in M13 phage for display with pIII, having the basic structure: signal sequence—variable region—Tyr-Gly-Gly-Phe-Leu—pIII. Phage from the library are then exposed to factor Xₐ and then screened on an antibody (e.g., 3E7), which is specific for N-terminally exposed Tyr-Gly-Gly-Phe-Leu. A pre-cleavage screening step with 3E7 can be employed to eliminate clones cleaved by E. coli proteases. Only members of the library with random sequences compatible with cleavage with factor Xₐ are isolated after screening, which sequences mimic the Ile-Glu-Gly-Arg site.

Another approach to protease substrate identification involves placing the variable region between the carrier protein and a reporter sequence that is used to immobilize the complex (e.g., Tyr-Gly-Gly-Phe-Leu). Libraries are immobilized using a receptor that binds the reporter sequence (e.g., 3E7 antibody). Phage clones having sequences compatible with cleavage are released by treatment with the desired protease.

To facilitate identification of the novel enterokinase recognition sequences of the present invention, a ligand recognition sequence, such as, for example SEQ ID NO:5 may be included in the phage library as a fusion partner attached to the potential EK recognition sequence. According to this method, the streptavidin binding peptide (e.g., SEQ ID NO:5) is expressed on the surface of the coat protein along with the enterokinase cleavage sequence. The resulting constructs, which have the basic structure: phage-EK recognition sequence-streptavidin binding peptide, are then bound to streptavidin (or avidin) through the streptavidin binding peptide moiety. The streptavidin may be immobilized on a surface such as a microtiter plate or on an affinity column. Alternatively, the streptavidin may be labeled, for example with a fluorophore, to tag the active phage peptide for detection and/or isolation by sorting procedures, e.g., on a fluorescence-activated cell sorter.

Phage which express peptides without the desired specificity are removed by washing. The degree and stringency of washing required will be determined for each ligand/enterokinase recognition sequence. A certain degree of control can be exerted over the binding characteristics of the peptides to be recovered by adjusting the conditions of the binding incubation and the subsequent washing or alternatively, as disclosed herein, by modifying the recognition sequences to increase their cleavage efficiency or rate.

Once a peptide sequence that imparts some affinity and specificity for the ligand binding partner is known, the diversity around this core sequence may be varied to affect binding affinity. For instance, variable peptide regions may be placed on one or both ends of the identified sequence. The known sequence may be identified from the literature, as in the case of Arg-Gly-Asp and the integrin family of receptors, for example, as described in Ruoslahti and Pierschbacher, Science, 238: 491497 (1987), or may be derived from earlier rounds of screening, as in the context of the present invention.

Since a useful enterokinase recognition sequence is already known, namely (Asp)₄-Lys-Xaa (SEQ ID NO:8), where Xaa is Ile in the native trypsinogen site or is any amino acid when incorporated in a synthetic EK-cleavable fusion protein, a practical standard for screening a phage display library for novel enterokinase recognition sequences was presented, in that cleavage sequences that were less specific or had a rate of cleavage only comparable to or slower than (Asp)₄-Lys-Xaa would be less desirable. Accordingly, although many novel enterokinase cleavage sequences may be discovered by the methods outlined above, we concentrated on isolation of enterokinase cleavage sequences providing advantages in comparison to (Asp)₄-Lys-Xaa (SEQ ID NO:8).

Synthesis of Peptides

Following the procedures outlined above, the synthetic polynucleotides coding for novel enterokinase recognition sequences expressed in recombinant phage recovered from the screening process may be isolated and sequenced, revealing the encoded amino acid sequences. After analysis of the recognition sequences to identify potential consensus sequences, recognition motifs, or recognition domains, it is desirable to vary these sequences to evaluate them as potential additional enterokinase recognition sequences. By chemically synthesizing peptide sequences of predetermined sequence and length, additional enterokinase recognition sequences may be evaluated and there is a strong possibility of identifying additional sequences with specificity and cleavage rates that are better than the isolates identified from the original phage library.

Synthesis may be carried out by methodologies well known to those skilled in the art (see, Kelley et al. in Genetic Engineering Principles and Methods, (Setlow, J. K., ed.), Plenum Press, NY., (1990) vol. 12, pp. 1-19; Stewart et al., Solid-Phase Peptide Synthesis (1989), W.H. Freeman Co., San Francisco) incorporated herein by reference. The enterokinase recognition sequences of the present invention can be made either by chemical synthesis or by semisynthesis. The chemical synthesis or semisynthesis methods allow the possibility of non-natural amino acid residues to be incorporated.

Enterokinase recognition peptides of the present invention are preferably prepared using solid phase peptide synthesis (Merrifield, J. Am. Chem. Soc., 85: 2149 (1963); Houghten, Proc. Natl. Acad. Sci. USA, 82: 5132 (1985)) incorporated herein by reference. Solid phase synthesis begins at the carboxy-terminus of the putative peptide by coupling a protected amino acid to a suitable resin, which reacts with the carboxy group of the C-terminal amino acid to form a bond that is readily cleaved later, such as a halomethyl resin, e.g., chloromethyl resin and bromomethyl resin, hydroxymethyl resin, aminomethyl resin, benzhydrylamine resin, or t-alkyloxycarbonyl-hydrazide resin. After removal of the α-amino protecting group with, for example, trifluoroacetic acid (TFA) in methylene chloride and neutralizing in, for example, TEA, the next cycle in the synthesis is ready to proceed. The remaining α-amino and, if necessary, side-chain-protected amino acids are then coupled sequentially in the desired order by condensation to obtain an intermediate compound connected to the resin. Alternatively, some amino acids may be coupled to one another forming an oligopeptide prior to addition of the oligopeptide to the growing solid phase polypeptide chain.

The condensation between two amino acids, or an amino acid and a peptide, or a peptide and a peptide can be carried out according to the usual condensation methods such as azide method, mixed acid anhydride method, DCC (dicyclohexylcarbodiimide) method, active ester method (p-nitrophenyl ester method, BOP [benzotriazole-1-yl-oxy-tris (dimethylamino) phosphonium hexafluorophosphate] method, N-hydroxysuccinic acid imido ester method), and Woodward reagent K method.

Common to chemical synthesis of peptides is the protection of the reactive side-chain groups of the various amino acid moieties with suitable protecting groups at that site until the group is ultimately removed after the chain has been completely assembled. Also common is the protection of the α-amino group on an amino acid or a fragment while that entity reacts at the carboxyl group followed by the selective removal of the α-amino-protecting group to allow subsequent reaction to take place at that location. Accordingly, it is common that, as a step in the synthesis, an intermediate compound is produced which includes each of the amino acid residues located in the desired sequence in the peptide chain with various of these residues having side-chain protecting groups. These protecting groups are then commonly removed substantially at the same time so as to produce the desired resultant product following purification.

The typical protective groups for protecting the α- and ε-amino side chain groups are exemplified by benzyloxycarbonyl (Z), isonicotinyloxycarbonyl (iNOC), O-chlorobenzyloxycarbonyl [Z(NO₂)], p-methoxybenzyloxycarbonyl [Z(OMe)], t-butoxycarbonyl (Boc), t-amyioxycarbonyl (Aoc), isobornyloxycarbonyl, adamatyloxycarbonyl, 2-(4-biphenyl)-2-propyloxycarbonyl (Bpoc), 9-fluorenylmethoxycarbonyl (Fmoc), methylsulfonyiethoxycarbonyl (Msc), trifluoroacetyl, phthalyl, formyl, 2-nitrophenylsulphenyl (NPS), diphenylphosphinothioyl (Ppt), dimethylophosphinothioyl (Mpt), and the like.

As protective groups for the carboxy group there can be exemplified, for example, benzyl ester (OBzl), cyclohexyl ester (Chx), 4-nitrobenzyl ester (ONb), t-butyl ester (Obut), 4-pyridylmethyl ester (OPic), and the like. It is desirable that specific amino acids such as arginine, cysteine, and serine possessing a functional group other than amino and carboxyl groups are protected by a suitable protective group as occasion demands. For example, the guanidino group in arginine may be protected with nitro, p-toluenesulfonyl, benzyloxycarbonyl, adamantyloxycarbonyl, p-methoxybenzenesulfonyl, 4-methoxy-2,6-dimethylbenzenesulfonyl (Mds), 1,3,5-trimethylphenysulfonyl (Mts), and the like. The thiol group in cysteine may be protected with p-methoxybenzyl, triphenylmethyl, acetylaminomethyl ethylcarbamoyl, 4-methylbenzyl, 2,4,6-trimethy-benzyl (Tmb), etc., and the hydroxyl group in the serine can be protected with benzyl, t-butyl, acetyl, tetrahydropyranyl, etc.

After the desired amino acid sequence has been completed, the intermediate peptide is removed from the resin support by treatment with a reagent, such as liquid HF and one or more thio-containing scavengers, which not only cleaves the peptide from the resin, but also cleaves all the remaining side-chain protecting groups. Following HF cleavage, the protein sequence is washed with ether, transferred to a large volume of dilute acetic acid, and stirred at pH adjusted to about 8.0 with ammonium hydroxide. Upon pH adjustment, the polypeptide takes its desired conformational arrangement.

Polypeptides according to the invention may also be prepared commercially by companies providing peptide synthesis as a service (e.g., BACHEM Bioscience, Inc., King of Prussia, Pa.; Quality Controlled Biochemicals, Inc., Hopkinton, Mass.).

Preparation of Fusion Proteins

According to the present invention, the novel enterokinase recognition sequences may be used to isolate and purify a protein of interest or a fragment thereof. By this method, the protein of interest is present as one domain of a recombinant fusion protein also including a novel enterokinase recognition sequence according to the present invention as another domain. Preferably, the first amino acid of the protein of interest is linked C-terminal to the EK cleavage sequence, and most preferably the N-terminal amino acid of the protein of interest takes the P₁′ position of the enterokinase recognition sequence. In this way, cleavage by enterokinase will separate the protein of interest exactly at the initial amino acid residue, avoiding any necessity of further treatment to remove extraneous N-terminal amino acids from the protein of interest.

The novel EK recognition sequence is also preferably ligated at its amino-terminal end to a ligand recognition sequence as the third domain of a fusion protein, facilitating immobilization to a ligand binding partner, such as, for instance, streptavidin.

A fusion protein is constructed using DNA manipulations according to conventional methods of genetic engineering (see, Sambrook J., Fritsch, E. F. and Maniatis T., Molecular Cloning: A Laboratory Manual, Cold Spring Harbor Laboratory Press, Cold Spring Harbor, N.Y. 1989). The preferred arrangement of the domains of a fusion protein designed for the recovery of the protein of interest will be (moving from N-terminal to C-terminal): a ligand recognition sequence, an enterokinase recognition sequence and a protein of interest. In constructing the preferred fusion protein of the present invention, a polynucleotide coding for the ligand recognition sequence is joined 5′ and in frame to a polynucleotide coding for an enterokinase recognition sequence, which, in turn, is linked 5′ and in frame to a polynucleotide coding for the protein of interest. Preferably, the codon for the N-terminal amino acid of the protein of interest will be positioned so as to take the P₁′ position (i.e., just C-terminal to the scissile bond of the EK cleavage sequence) in the fusion protein construct. The fusion protein expression constuct will also typically include a promoter for directing transcription in a selected host, a ribosome binding site, and a secretion signal peptide for directing secretion of the fusion protein from a transformed host cell.

The plasmid containing the nucleotides coding for the fusion protein of the present invention may be constructed by ligating the DNA fragments into an expression vector of choice by techniques well known in the art. For the construction, conventional DNA ligation techniques may be used. For instance, using the restriction enzyme method, the nucleotide sequences which comprise the sequences that are translated into the fusion protein, after isolation and/or synthesis, may be restriction digested at strategic sites to create DNA sequence overhangs as a template for fusion to another DNA molecule having an homologous overhang or sequence. Alternatively, a single-stranded DNA overhang may be synthetically constructed onto a DNA fragment that either has an existing overhang or is blunt-ended by using techniques well known in the art. The homologous, single-stranded DNA overhangs of each nucleotide sequence are then ligated using a commercially available ligase such as, for instance, T4 DNA ligase, to create a fused DNA fragment comprising DNA from different regions of the same organism or DNA from different organisms or sources. Theoretically, the only limitation to the number of DNA fragments that may be ligated or the size of the ligated fragment is limited by the size of the fragment that can be inserted into the vector or expression vector of choice.

By a similar method, the fused DNA fragments are then ligated into an expression vector which has been treated with the appropriate restriction enzyme or enzymes to create a splice site within the vector that is compatible with the 5′ and 3′ ends of the DNA fragment to be inserted for expression. After ligation is complete, the recombinant vector is introduced into the appropriate host cell for expression of the protein of interest fused with the ligand recognition and enterokinase recognition sequences.

Isolation and Purification of a Protein of Interest

For expression of the fusion protein, cells transformed with the expression vector are grown in cell culture under conditions suitable for the expression of the protein of interest. After expression the cells may be lysed to release the fusion protein into the cell culture or preferably the fusion protein will include a signal sequence to facilitate secretion of the fusion protein into the culture medium without the need for disruption or lysis of the host cell. Secretion of the fusion protein into the culture medium is preferred, as the fusion protein may be isolated directly from the culture supernatant. If the cells require lysis, one or more additional purification steps will be necessary to separate the fusion protein from the cellular debris released upon lysis of the cells. This may result in reduced yields of the protein of interest or a diminution of its biological activity.

The fusion proteins of the present invention may be isolated and purified by standard methods including chromatography (e.g., ion exchange, affinity, sizing column chromatography, and high pressure liquid chromatography), centrifugation, differential solubility, or by any other standard technique for the purification of proteins.

In one aspect of the invention, large quantities of the fusion protein may be isolated and purified by passing the cell culture supernatant containing the expressed fusion protein over a column containing an immobilized ligand binding partner specific for the ligand recognition sequence included in the fusion protein construct, such as, for example, streptavidin (i.e., where the fusion protein contains a biotin or other streptavidin binding domain). After binding, the column is washed to remove any unbound fusion peptides. Following the wash step, the column is contacted with enterokinase under incubation conditions and enzyme concentrations suitable for cleavage of the enterokinase recognition sequence. The released protein is then eluted and recovered in substantially pure and biologically active form by standard methods known in the art. In most instances the recovered protein of interest will not require any further purification steps. Alternatively, enterokinase may be added to the culture medium prior to contacting the culture media with a ligand binding partner so as to isolate or immobilize the binding partner/EK cleavage sequence portion of the fusion protein and leave the protein of interest portion in solution.

The present invention may be further illustrated by reference to the following non-limiting examples.

### EXAMPLES

### Construction and Screening of Phage Display Library for EK Cleavage Sequences

(i) Construction of Substrate Phage Library

A phage display library was designed for the display of an exogenous polyeptide at the N-terminus of M13 phage gene III protein. The exogenous polyeptide was an 86-mer fusion protein having tandem ligand recognition sequences, a variegated segment of thirteen amino acids serving as a template for potential EK recognition sequences, a factor Xa cleavage site, segments linking the foregoing domains and linking to the N-terminus of gene III protein. The sequence of the exogenous display polypeptide was as follows:

| | |
|---|---|
| AEWHPQFSSPSASRPSEGPCHPQFPRCYIENLDEFR | (SEQ ID NO: 9) |
| PGGSGGXXXXXXXXXXXXXGAQSDGGGSTEHAEGGS | |
| ADPSYIEGRIVGSA-(gene III protein | |
| N-terminus), | |

wherein any amino acid residue except cysteine was permitted at each X position. The underscored segments denote, moving from N-terminal to C-terminal, a linear streptavidin binding sequence, a constrained streptavidin binding loop, and a factor Xa cleavage site, respectively. This design gave a potential diversity of 4.2×10¹⁶. Approximately 2×10⁸ different display polypeptides were included in the library for screening.
(ii) Screening Library for Novel Enterokinase Cleavage Sequences

The substrate phage library having a diversity of 2×10⁸ display polypeptide sequences was screened for phage that could be released by enterokinase cleavage after binding to streptavidin immobilized on polystyrene magnetic beads.

Phage were screened for a total of five rounds. In each screening round, two aliquots of phage were allowed to bind streptavidin beads in separate tubes by incubation at room temperature for 30 minutes in EK assay buffer (20 mM Tris-HCl, pH 7.4, 50 mM NaCl, 2 mM CaCl₂, 0.05% Triton X-100). After washing with EK assay buffer (500 μL×5), the bead bound phage were incubated with recombinant light chain enterokinase (Novagen, Madison, Wis.) in assay buffer at room temperature.

DNA sequence analysis of up to 40 randomly chosen phage isolates from each screening condition was performed at round 2 and all subsequent rounds to monitor the progress of substrate selection. The stringency of screening conditions was increased in rounds 4 and 5 as consensus sequence patterns were not clearly discernible after round 3.

In rounds 1 thru 3, two different enterokinase concentrations were used. The 320 nM susceptible phage populations were treated consistently at 320 nM enterokinase in all three rounds and the 1.3 μM enterokinase susceptible phage populations were treated consistently at that concentration in all three rounds.

In round 4, the 320 nM enterokinase susceptible phage from round 3 were bound to streptavidin beads then incubated for 30 minutes with 65 nM enterokinase in enterokinase assay buffer. The beads were pelleted by centrifugation for 30 sec in a microfuge and the supernatant containing the enterokinase-cleaved phage was removed. Fresh 65 nM enterokinase in assay buffer was added to the beads for an additional 1.5 hr incubation to cleave remaining phage.

For round 5, two aliquots of the 30 minute enterokinase-susceptible phage from round 4 were bound to separate batches of streptavidin beads for incubation in either 10 nM enterokinase or 30 nM enterokinase.

After removing the “cleaved” phage supernatants from the streptavidin beads in each round, the supernatants were mixed with two successive batches of fresh streptavidin beads for 30 minutes at room temperature to eliminate any free phage that retained the streptavidin binding domain. The final unbound phage supernatants were used to infect host Escherichia coli cells to amplify the phage populations for each subsequent round of screening.

The amplified phage populations from round 5 were tested for enterokinase cleavage by phage ELISA. Round 5 phage populations were screened against phage from the unselected substrate library as a negative control.

Individual phage samples were allowed to bind to streptavidin-coated microtiter wells and then subjected to different concentrations of enterokinase for 2 hours at room temperature. Unreleased phage were detected using an anti-phage antibody-horseradish peroxidase (HRP) conjugate and HRP activity assay. The decline in absorbance at 630 nm in streptavidin-bound phage with increasing enterokinase concentrations observed for the round 5 phage populations indicated successful selection for enterokinase substrates.

(iii) Identification of Specific Enterokinase Cleavage Sequences

The DNA sequences of 82 of the 90 randomly chosen phage isolates from round 5, when grouped by sequence similarity, yielded a simple acidic amino acid-basic amino acid double codon motif that included a 66% frequency of the codon sequence for Asp-Arg, 14% for Glu-Arg, 18% for Asp-Lys, and 4% for Glu-Lys. The sequences from isolation rounds 2-4 were reviewed for the acid-base motif, and the single EK cleavage site peptide substrates are set forth in Tables 1, 2 and 3. Hexamers upstream (N-terminal) with respect to the scissile bond (P_{1′}) were noted, as this peptide length was regarded as indicative of a high specificity substrate. The peptides are listed as heptamers including the P_{1′} amino acid residue. Amino acid residues in bold type are from the variegated region of the display peptide; amino acid residues depicted in regular type are constant residues from the phage protein.

TABLE 1

Amino Acid Sequences of Round 2 Isolates

| | Amino | SEQ |
|---|---|---|
| Isolate | Acid Sequence | ID NO: |
| 02-A01 | Y E W Q D R T | 10 |
| 02-A03 | N S I K D R V | 11 |
| 02-A07 | A K A T E R H | 12 |
| 02-A09 | L G K V D R T | 13 |
| 02-A10 | G G M A D K F | 14 |
| 02-B05 | G H W L D K N | 15 |
| 02-B07 | N K A K D R M | 16 |
| 02-B11 | S E N F D K N | 17 |
| 02-C03 | L D W E D R A | 18 |
| 02-C04 | S T D A E R M | 19 |
| 02-C05 | H T F S D R Q | 20 |
| 02-C07 | G S G G D R L | 21 |
| 02-C09 | G F Y N D R M | 22 |
| 02-C10 | I M P Q D K S | 23 |
| 02-C11 | G G V E D R S | 24 |
| 02-D03 | W Q E S D R A | 25 |
| 02-E02 | G S G G D R H | 26 |
| 02-F06 | G H I F D R S | 27 |
| 02-E02 | G S G G E K L | 28 |
| 02-F01 | S G G E D R M | 29 |
| 02-F02 | G S G G E R T | 30 |
| 02-F05 | P D P Q E R Q | 31 |
| 02-F06 | Y I M G D R T | 32 |
| 02-F07 | Q N H S D R T | 33 |
| 02-F08 | I A H G E R A | 34 |
| 02-F12 | H E M N D R H | 35 |
| 02-G01 | T H N G E K M | 36 |
| 02-G02 | H D E A E K T | 37 |
| 02-G04 | G Y W I D R S | 38 |
| 02-G05 | G S G G E R L | 39 |
| 02-G06 | S G G S D R L | 40 |

TABLE 2

Amino Acid Sequences of Round 3 Isolates

| | Amino | SEQ |
|---|---|---|
| Isolate | Acid Sequence | ID NO: |
| 03-A02 | A Q Y M D L M | 41 |
| 03-A03 | G S G G E R N | 42 |
| 03-A04 | G S G G E N G | 43 |
| 03-A06 | E N Y E E R T | 44 |
| 03-A07 | N I Y G D R I | 45 |
| 03-A12 | G G F V D K Q | 46 |
| 03-B01 | G S G G E K V | 47 |
| 03-B04 | G K F E D R N | 48 |
| 03-B08 | P A H T D R D | 49 |
| 03-B09 | Q Q M H D R F | 50 |
| 03-B12 | D M G Y D R G | 51 |
| 03-C02 | S G G D E K E | 52 |
| 03-C04 | I E S A D R T | 53 |
| 03-C11 | R N M D E R A | 54 |
| 03-D03 | T V G M D K F | 55 |
| 03-D10 | G S G G D R F | 56 |
| 03-D11 | R H N Y D R I | 57 |
| 03-D12 | V Y H V D K M | 58 |
| 03-E01 | G S G G E R N | 59 |
| 03-F01 | G G K Y D R M | 60 |
| 03-G01 | G G N D D K M | 61 |
| 03-H02 | A A V E D R N | 62 |
| 03-H05 | P C K D E R F | 63 |
| 03-H12 | G S E L D R M | 64 |

TABLE 3

Amino Acid Sequences of Round 4 Isolates

| | Amino | SEQ |
|---|---|---|
| Isolate | Acid Sequence | ID NO: |
| 04-A01 | F S E E D R M | 65 |
| 04-A03 | G S G G E R F | 66 |
| 04-A04 | Y Q P T D R T | 67 |
| 04-A05 | S G G E D R M | 68 |
| 04-A06 | T E Q M D R M | 69 |
| 04-A07 | Q P F D D R D | 70 |
| 04-A08 | G S G G E R T | 71 |
| 04-A09 | E G M T D R L | 72 |
| 04-A10 | E I P E D R M | 73 |
| 04-A11 | G D D D D K I | 74 |
| 04-B02 | G S G G E R S | 75 |
| 04-B03 | H G Y E E R M | 76 |
| 04-B05 | K P M E E R M | 77 |
| 04-B06 | S G G N D R M | 78 |
| 04-B07 | G G T D D R F | 79 |
| 04-B08 | D V Y S E R M | 80 |
| 04-B12 | D V Y S E R M | 81 |
| 04-C01 | G S G G D R N | 82 |
| 04-C02 | D V T A D D R | 83 |
| 04-C04 | A E F A D R F | 84 |
| 04-C06 | N N S D E K I | 85 |
| 04-C08 | P G G D D R W | 86 |
| 04-C09 | S G G E E R V | 87 |
| 04-C10 | V W P D D R S | 88 |
| 04-C11 | H R Q T D R M | 89 |
| 04-D02 | K E A E D R A | 90 |
| 04-D03 | V G D D E R H | 91 |
| 04-D04 | N S M A D R N | 92 |
| 04-D06 | T E F E D K W | 93 |
| O4-D07 | E S G G E R D | 94 |
| 04-D08 | N N Y W D R M | 95 |
| 04-D09 | F S E E D R M | 96 |
| 04-D11 | E M H E E R M | 97 |
| 04-D12 | D Q M E D R Q | 98 |
| 04-E01 | E W K M D R M | 99 |
| 04-E02 | S Y T W D R S | 100 |
| 04-E03 | S F M L D R M | 101 |
| 04-E05 | T E V D D R H | 102 |
| 04-E06 | G D Q E D R M | 103 |
| 04-E07 | H N I D D R I | 104 |
| 04-E08 | A S W E D R T | 105 |
| 04-E09 | G G E D D R S | 106 |
| 04-E10 | D I Q D E R N | 107 |
| 04-F01 | D T H A D K S | 108 |
| 04-F02 | G S G G D R M | 109 |
| 04-F03 | G E I M D R S | 110 |
| 04-F05 | G S G G D K T | 111 |
| 04-F06 | G S G G D R A | 112 |
| 04-F07 | G D H L D R H | 113 |
| 04-F08 | G Q Q D D R Q | 114 |
| 04-F09 | A L A A D R M | 115 |
| 04-F10 | V G F D D R T | 116 |
| 04-F11 | Y A Q D E R T | 117 |
| 04-F12 | G G R E E R N | 118 |
| 04-G02 | G S G G D R M | 119 |
| 04-G04 | G S G G D R E | 120 |
| 04-G05 | I A Y Q D R M | 121 |
| 04-G08 | S G G E D R A | 122 |
| 04-G09 | L E H S D R V | 123 |
| 04-G10 | F K P D D R M | 124 |
| 04-G11 | V P M A D R S | 125 |
| 04-G12 | G S G G E R A | 126 |
| 04-H02 | N D N D E R A | 127 |
| 04-H04 | G N Y T D R M | 128 |
| 04-H05 | G S G G E R V | 129 |
| 04-H06 | D E V H D R T | 130 |
| 04-H07 | Q H D G D K T | 131 |
| 04-H08 | T V R S E K G | 132 |
| 04-H10 | S G G T D R I | 133 |

The sequenced Round 5 EK recognition sequences having at least three amino acids from the variegated region N-terminal to the scissile bond are shown in Table 4. Sequences having more than one acid-base combination (and thus being suspected of encompassing a double cleavage site) or no acid-base combination are eliminated from the table. The hexamer including the acid-base combination and the amino acid C-terminal to the scissile bond are shown. The EK cleavage substrate was regarded as being defined by three to six amino acids upstream (N-terminal) of the scissile bond.

TABLE 4

Amino Acid Sequences of Round 5 Isolates

| | Amino | SEQ |
|---|---|---|
| Isolate | Acid Sequence | ID NO: |
| 05-A02 | V M E D D R A | 134 |
| 05-A03 | G S G G E R M | 135 |
| 05-A05 | I E H D D R M | 136 |
| 05-A08 | F S E E D R M | 137 |
| 05-A10 | F S E E D R M | 138 |
| 05-A11 | D V Y S E R M | 139 |
| 05-A12 | D M F D D R M | 140 |
| 05-B01 | F S E E D R M | 141 |
| 05-B02 | E H L F D R M | 142 |
| 05-B03 | S W I S D R V | 143 |
| 05-B04 | N D E D D R M | 144 |
| 05-B05 | S L D D D R T | 145 |
| 05-B06 | G S G G D R D | 146 |
| 05-B08 | P H I E D R M | 147 |
| 05-B09 | S G G D D R H | 148 |
| 05-B10 | E V F A D R S | 149 |
| 05-B11 | G L A E D R T | 150 |
| 05-C01 | S G G D D R L | 151 |
| 05-C04 | S G G D D R M | 152 |
| 05-C05 | G L V S E R G | 153 |
| 05-C08 | G G F E D K M | 154 |
| 05-C09 | S L D D D R T | 155 |
| 05-C10 | D V Y S E R M | 156 |
| 05-D01 | N M D W D R S | 157 |
| 05-D02 | S L D D D R T | 158 |
| 05-D03 | G S G G D R M | 159 |
| 05-D05 | F S E E D R M | 160 |
| 05-D07 | S L D D D R T | 161 |
| 05-D09 | V D M H D R M | 162 |
| 05-D10 | S G G D D R M | 163 |
| 05-D12 | N V R M D R S | 164 |
| 05-E02 | S H R D E K V | 165 |
| 05-E03 | L M N D D R A | 166 |
| 05-E05 | F V M N D K G | 167 |
| 05-E06 | V S D D D R A | 168 |
| 05-E07 | G H V D D R M | 169 |
| 05-E08 | H A I E E R S | 170 |
| 05-E10 | D I N D D R S | 171 |
| 05-E11 | G S G G E R T | 172 |
| 05-E12 | A V I G D R S | 173 |
| 05-F01 | S G G E E R G | 174 |
| 05-F05 | V E F Y D R M | 175 |
| 05-F09 | G S G G E R I | 176 |
| 05-F11 | S L D D D R T | 177 |
| 05-G02 | S G G Q E R S | 178 |
| 05-G03 | D I N D D R S | 179 |
| 05-G04 | D H V W D R A | 180 |
| 05-G05 | G S G G D R I | 181 |
| 05-G06 | I E D E D R A | 182 |
| 05-G07 | M T F D E R G | 183 |
| 05-G08 | G D W D D K N | 184 |
| 05-G09 | I A Y Q D R M | 185 |
| 05-G11 | G S G G D R I | 186 |
| 05-G12 | G F V Q E R M | 187 |
| 05-H04 | D I N D D R S | 188 |
| 05-H05 | G W N D D R I | 189 |
| 05-H06 | G G F E D R L | 190 |
| 05-H08 | G S G G D R N | 191 |
| 05-H09 | A A V E D R N | 192 |
| 05-H10 | D Y R L D R I | 193 |
| 05-H11 | G D D D D K I | 194 |

The five sequences that occurred in the selected phage more than once are shown in Table 5, below. Interestingly, only one instance of the native enterokinase substrate sequence (Asp)₄-Lys-Ile was identified (05-H11).

TABLE 5

Amino acid sequences of EK
recognition sequences from Substrate
Phage Library Isolates that occurred
more than once among 82 sequenced isolates

| phage | variable | | SEQ |
|---|---|---|---|
| isolate | region sequence | frequency | ID NO: |
| 5-A01 | DRMYQLDKTGFMI | 11 | 195 |
| 5-A08 | DMFSEEDRMMQMQ | 4 | 137 |
| 5-A11 | DLNDVYSERMAMW | 2 | 139 |
| 5-B05 | SLDDDRTVSPKFW | 5 | 145 |
| 5-H04 | DINDDRSLFSESS | 3 | 188 |
| 5-H11 | MGDDDDKIYVYKT | 1 | 194 |
| 5-F08 | AVLSNVMHSDDWT | unselected | 196 |
| | | control | |

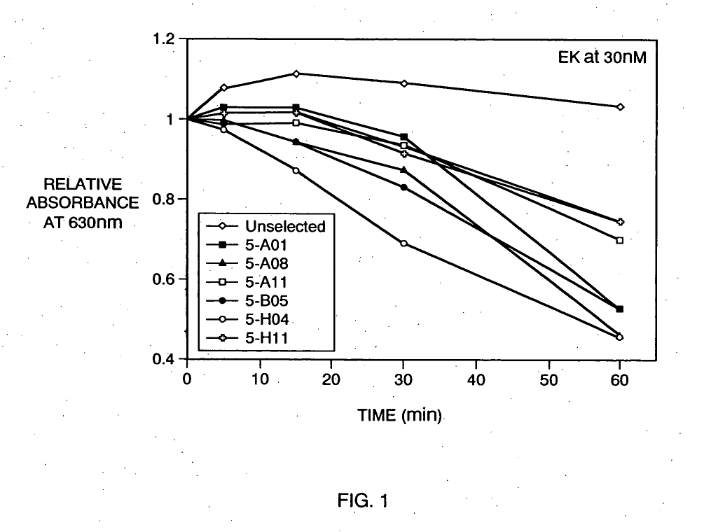

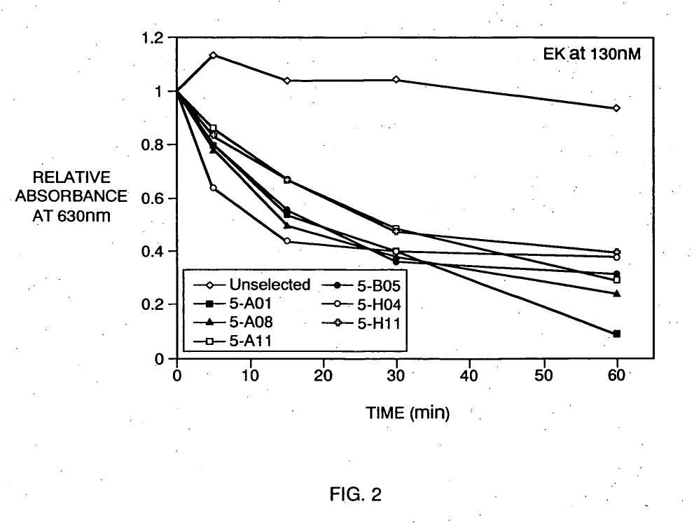

Phage displaying each of the sequences shown in Table 5 were tested individually for kinetics of enterokinase cleavage using a phage ELISA. Streptavidin-bound phage were treated with either 30 nM or 130 nM enterokinase for 30 minutes. The time courses of phage release are shown in FIG. 1 (release at 30 nM EK) and FIG. 2 (release at 130 nM EK). Phage from the unselected substrate library were used as a control, i.e., isolate 5-F08. (SEQ ID NO:196).

The kinetics of enterokinase cleavage differed between the two concentrations of enterokinase used. At 30 nM enterokinase, there was a lag in phage release which was not observed at 130 nM enterokinase. This may be attributed to a requirement for the enzyme to cut three to five copies of the substrate peptide on a single phage for successful release.

In comparing the enterokinase cleavage rates of each phage type, isolate 5-H04 (SEQ ID NO:188) shown in Table 5 was the most readily cut, and the cleavage rate for the (Asp)₄-Lys-containing recognition sequence 5-H11 (SEQ ID NO:194) was slower than for at least three of the other isolates, i.e., 5-A08 (SEQ ID NO:137), 5-B05 (SEQ ID NO:145) and 5-H04 (SEQ ID NO:188).

(iv) Comparative Analysis of Preferred Enterokinase Cleavage Sites

To further test the predicted cleavage site as well as the rates and extent of cleavage, seven test peptides shown in Table 6 were chemically synthesized, contacted with enterokinase, and analyzed by HPLC and mass spectrometric analysis.

TABLE 6

Synthetic Test Peptides

| test peptide sequence | SEQ |
|---|---|
| ↑ = predicted cleavage site | ID NO: |
| GDDDDK↑IYV (positive control) | 197 |
| AVLSNVMFI (negative control) | 198 |
| GNYTDR↑MFI | 199 |
| DINDDR↑SLF | 200 |
| NKAKDR↑MFI | 201 |
| GNYTDR↑RFI | 202 |
| GNYTDR↑YFI | 203 |

To test the predicted cleavage site, i.e., following the acid-base dipeptide motif, 60 to 100 μg of each test peptide was digested to completion (36-48 hrs) with 20U of recombinant light chain enterokinase (Novagen) and analyzed by reverse phase HPLC. Product peaks were eluted with a water/acetonitrile (H₂O/ACN) gradient and identified by electrospray mass spectroscopy. The results of the cleavage test are shown in Table 7.

TABLE 7
EK Cleavage Products

| Test Peptide | product peak | recovered product | % ACN |
|---|---|---|---|
| GDDDDK_{↑}IYV | 1 | -- | |
| | 2 | IYV | 20 |
| AVLSNVMFI | 1 | -- | |
| | 2 | -- | |
| GNYTDR_{↑}MFI | 1 | GNYTDR | 9 |
| | 2 | MFI | 23 |
| DINDDR_{↑}SLF | 1 | DINDDR | 8 |
| | 2 | SLF | 21 |
| NKAKDR_{↑}MFI | 1 | -- | |
| | 2 | MFI | 23 |
| GNYTDR_{↑}RFI | 1 | GNYTDR | 9 |
| | 2 | RFI | 17 |
| GNYTDR_{↑}YFI | 1 | GNYTDR | 9 |
| | 2 | YFI | 22 |

HPLC demonstrated that all digestions were carried to completion (except for the negative control which was not cleaved at all). “% ACN” estimates the position in the H₂O/Acetonitrile gradient at which the indicated cleavage fragment eluted. The expected product peaks for GDDDDK (residues 1-6, SEQ ID NO:197) and NKAKDR (residues 1-6, SEQ ID NO:201) were not detected by HPLC, but the cleavage site could be determined from analyzing the alternate product peak, i.e., the peptide to the C-terminal side of the cleavage site. Results demonstrated that in all cases, enterokinase-catalyzed hydrolysis of the peptide bond occurred at the anticipated position. (See arrows in Table 6.) No cleavage occurred with the negative control peptide (SEQ ID NO:198).

(v) Relative Rate of Cleavage

Peptides were digested with enterokinase and aliquots tested at timed intervals by HPLC to quantitate the extent of cleavage. For each test peptide, about 500 μM of peptide were digested with 50 nM of recombinant light chain enterokinase. The seven synthetic peptides were compared with a commercially available standard EK cleavage substrate, GDDDDK-β naphthylamine (GDDDDK-βNA, SEQ ID NO:203; from BACHEM, King of Prussia, Pa.), having a fluorescent leaving group that increases in fluorescence when it is cleaved. The molar rates of substrate cleavage are shown in Table 8.

TABLE 8
Relative Rates of Cleavage

| | Cleavage Rate | rate relative to |
|---|---|---|
| Test Peptide | (nmole/min.) | standard substrate |
| GDDDDK-βNA | 0.46 | (1.0) |
| GDDDDKIYV | 0.34 | 0.7 |
| GNYTDRMFI | 0.81 | 1.8 |
| DINDDRSLF | 1/43 | 3.1 |
| NKAKDRNFI | 0.26* | 0.6 |
| GNYTDRRFI | 0.18 | 0.4 |
| GNYTDRYFI | 0.24 | 0.5 |

*results estimated due to peak overlap

Peptides GNYTDRMFI (SEQ ID NO:199) and DINDDRSLF (SEQ ID NO:200) were cleaved significantly more rapidly than the two control peptides that included the native enterokinase recognition sequence, i.e., GDDDDKIYV (SEQ ID NO:197) and GDDDDK-βNA (SEQ ID NO:203). These two control peptides were cleaved at nearly equal rates and more rapidly than the remaining three peptides tested.

(vi) Substrate Competition with Reference Peptide

Rates of substrate hydrolysis depends on several factors, namely, concentration of enzyme and substrate, Kₘ (Michaelis constant) values, and k_{cat} (catalytic rate constant) values. One way to compare the relative efficiencies with which a protease hydrolyses two substrates (a and b) is to simultaneously incubate both substrates in a single reaction with the enzyme and measure the rates of product formation for each (Vₐ and V_{b}). If the total product formation is low (<10%), the starting concentrations of the two competing substrates are the same, and the reaction is performed at steady-state:
Vₐ/V_{b}=(K_{cat}/Kₘ)ₐ/(k_{cat}/Kₘ)_{b}
Relative ratios of K_{cat}/Kₘ can be determined from relative rates of substrate hydrolysis.

To compare the relative efficiency of hydrolysis by enterokinase, reference peptide (GDDDDK-βNA, 250 μM, SEQ ID NO:203) was incubated simultaneously with one of the test peptides (250 μM), treated with enterokinase, and the relative rate of product formation measured. The products were quantitated by HPLC and initial cleavage rates calculated. Table 9 shows the individual cleavage rates for each peptide and the relative ratio of test peptide cleavage rate to reference peptide cleavage rate.

TABLE 9
Relative Hydrolysis Rates in Competitive Assay

| | test peptide | reference peptide | ratio |
|---|---|---|---|
| Test Peptide | rate (Va) | rate (Vb) | (Va/Vb) |
| GDDDDKIYV | 0.028 | 0.027 | 1.0 |
| DINDDRSLF | 0.18 | 0.006 | 30 |
| GNYTDRMFI | 0.038 | 0.011 | 3.5 |

The results demonstrated that the peptide Asp-Ile-Asn-Asp-Asp-Arg-Xaa (SEQ ID NO: 204) serves as an excellent substrate for cleavage by enterokinase, where the scissile bond is between Arg and Xaa, and where Xaa can be any amino acid, e.g., the first amino acid residue of a polypeptide to be cleaved from the substrate. The cleavage rate of the test peptide including SEQ ID NO:204 was 3.1 times the rate of the reference peptide when tested individually at 500 μM. The ratio K_{cat}/Kₘ was 30 times greater than that of the reference peptide when tested in competition at 250 μM. The results further point to the substrate peptide Gly-Asn-Tyr-Thr-Asp-Arg-Xaa (SEQ ID NO:205) as superior to the known substrate (Asp)₄-Lys. The test peptide including SEQ ID NO:205 was 1.8 times the rate of the reference peptide when tested individually at 500 μM, and the ratio K_{cat}/Kₘ was 3.5 times greater than that of the reference peptide when tested in competition at 250 μM.

(vii) Identity of Residues on C-Terminal Side of Scissile Bond

Additional experiments were performed to test whether the discovered EK recognition substrates would show a preference for the identity of the amino acid in the P₁′ position, that is, at the position that would be the N-terminus of a polypeptide cleaved from the EK recognition substrate. The round 5 isolates were selected for the most efficient cleavage by enterokinase. While it is useful to determine which amino acids at the P₁′ position promote the most efficient cleavage by enterokinase, it is also important to know all the amino acids at the P₁′ position that promote any cleavage by enterokinase.

DNA sequencing of the phage isolates identified phage clones having 16 of the 20 amino acids at the P₁′ position following the Asp-Arg (DR) motif. Only four amino acids were not observed in any of the isolates at the P₁′ position following Asp-Arg, among those isolates sequenced: Lys, Pro, Arg and Cys (which was not permitted in the 13-mer variable portion when the substrate phage library was generated). The absence of any phage isolates exhibiting these amino acids at the P₁′ position does not mean that an EK recognition sequence such as Asp-Ile-Asn-Asp-Asp-Arg-Xaa (SEQ ID NO:204) having Lys, Pro, Arg or Cys at the Xaa position will not be cleaved; rather it indicates that such recognition sequences will be cleaved less efficiently than recognition sequences having the other amino acids at the Xaa (P₁′) position.

A phage ELISA assay was used to test examples of P₁′ residues for EK cleavage. 17 isolates from rounds 2-5 of screening and exhibiting the Asp-Arg motif before the scissile bond (P₂-P₁) were chosen for enterokinase cleavage analysis. Phage were bound to streptavidin immobilized in microtiter wells and then treated with either 100 nM or 300 nM recombinant light chain enterokinase for 30 minutes. For each isolate, ELISA signals obtained after entrokinase treatments were compared to the signal obtained in the absence of enterokinase treatment. Three negative controls were included: the unselected substrate phage library, isolate 5-F08 (SEQ ID NO:196) containing no cleavage sites, and a phage with an irrelevant but functional display peptide, having a thrombin cleavage site in place of the varied (13-mer) sequence.

The results showed that at the 100 nM concentration, phage displaying Met, Thr, Ser, or Ala residues at the P₁′ position were most sensitive to enterokinase treatment, phage displaying residues Asp, Leu, Phe, Asn, Trp, Ile, Gln, or Glu residues at the P₁′ position were less sensitive to 100 nM enterokinase treatment, and phage displaying residues His, Val, Gly, and Tyr at the P₁′ position were most resistant to enterokinase treatment. All of the phage isolates were readily cleaved when the enterokinase concentration was raised to 300 nM.

Analysis of the sequence information from screening Rounds 4 and 5 was performed to detect preferences for amino acids at the positions upstream of the scissile bond, in order to select preferred EK cleavage sequences. For the most numerous group, i.e., cleavage sequences having the Asp-Arg motif at the P₂ and P₁ positions, an amino acid was regarded as preferred at a given position in the sequence if it occurred in five or more isolates. Where a phage residue occurred at a given position, it was not counted. From this analysis, a family of preferred EK recognitions sequences was defined having the following formula:
Xaa₁-Xaa₂-Xaa₃-Xaa₄-Asp-Arg-Xaa₅ (SEQ ID NO:206),
wherein Xaa₁ is an optional amino acid residue which, if present, is Ala, Asp, Glu, Phe, Gly, Ile, Asn, Ser, or Val; Xaa₂ is an optional amino acid residue which, if present, is Ala, Asp, Glu, His, Ile, Leu, Met, Gln, or Ser; Xaa₃ is an optional amino acid residue which, if present, is Asp, Glu, Phe, His, Ile, Met, Asn, Pro, Val, or Trp; Xaa₄ is Ala, Asp, Glu, or Thr; and Xaa₅ can be any amino acid residue.

For the next most numerous group, i.e., cleavage sequences having the Glu-Arg motif at the P₂ and P₁ positions, an amino acid was regarded as preferred at a given position in the sequence if it occurred in four or more isolates. From this analysis, a family of preferred EK recognition sequences was defined having the following formula:

| | |
|---|---|
| Xaa₁-Xaa₂-Xaa₃-Xaa₄-Glu-Arg-Xaa₅, | (SEQ ID NO: 207) |

wherein Xaa₁ is an optional amino acid residue which, if present, is Asp or Glu; Xaa₂ is an optional amino acid residue which, if present, is Val; Xaa₃ is an optional amino acid residue which, if present, is Tyr; Xaa₄ is Asp, Glu, or Ser; and Xaa₅ can be any amino acid residue.

Analysis of the sequences from Rounds 24 having the other acid-base combinations, i.e. Asp-Lys and Glu-Lys at the P₂ and P₁ positions, did not reveal any preferences at any of the upstream positions P₃, P₄, P₅ or P₆.

Following the foregoing description, additional enterokinase cleavage sequences can be identified and synthesized, and utilized in fusion protein expression to simplify purification of any protein of interest. By following the procedures described herein, several novel cleavage sequences were discovered, and surprisingly two were tested that showed rates of cleavage several times that of the native EK recognition sequence of (Asp)₄-Lys-Ile (SEQ ID NO:8). Additional EK recognition sequences will become apparent to those skilled in the art following the teachings herein. For example, minor modifications to the EK cleavable recognition sequences disclosed herein may be made to improve ease of synthesis or some other property without eliminating EK recognition and without departing from the scope of this discovery.

Likewise, truncation of the preferred EK recognition sequences by substitution at positions distal from the scissile bond (e.g., sequences corresponding to amino acids 2-6 or 3-6 or 4-6 of SEQ ID NO:1) are expected to function as EK recognition sequences, although the specificity and rate of EK cleavage of a fusion protein including them may be vastly inferior to the preferred sequences disclosed above.

It will be understood by those skilled in the art that additional substitutions, modifications and variations of the described embodiments and features may be made without departing from the invention as described above or as defined by the appended claims.

The publications cited herein are hereby incorporated by reference in their entireties.

1. A polypeptide comprising an enterokinase recognition sequence and having the formula: Z₁-Xaa₁-Xaa₂-Xaa₃-Xaa₄-Asp-Arg-Xaa₅-Z₂ (SEQ ID NO:1), wherein
(i) Xaa₁ is an optional amino acid, which if present, is Ala, Asp, Glu, Phe, Gly, Ile, Asn, Ser, or Val; Xaa₂ is an optional amino acid which, if present, is Ala, Asp, Glu, His, Ile, Leu, Met, Gln or Ser; Xaa₃ is an optional amino acid residue which, if present, is Asp, Glu, Phe, His, Ile, Met, Asn, Pro, Val, or Trp; Xaa₄ is Ala, Asp, Glu, or Thr; and Xaa₅ can be any amino acid; and wherein Z₁ and Z₂ are both optional and are, independently, polypeptides of one or more amino acids.

2. The polypeptide of claim 1, wherein Xaa₁ is Asp, Xaa₂ is Ile, Xaa₃ is Asn, Xaa₄-is Asp, and Xaa₅-is Met, Thr, Ser, Ala, Asp, Leu, Phe, Asn, Trp, Ile, Gln, Glu, His, Val, Gly or Tyr.

3. The polypeptide of claim 1, wherein Z₁ is a ligand recognition sequence.

4. The polypeptide of claim 1, wherein Z₁ is a streptavidin binding domain.

5. The polypeptide of claim 4, wherein the streptavidin binding domain is selected from the sequences: His-Pro-Gln-Phe (SEQ ID NO:6), Cys-His-Pro-Gln-Phe-Cys (SEQ ID NO: 5), Cys-His-Pro-Gln-Phe-Cys-Ser-Trp-Arg (SEQ ID NO: 7), Trp-His-Pro-Gln-Phe-Ser-Ser (SEQ ID NO:210), Pro-Cys-His-Pro-Gln-Phe-Pro-Arg-Cys-Tyr (SEQ ID NO:211), and tandemly arranged combinations and repeats thereof.

6. (canceled)

7. The polypeptide of claim 1, wherein the polypeptide Xaa₅-Z₂ is a protein of interest.

8-12. (canceled)

13. The polypeptide of claim 1, comprising an enterokinase recognition sequence having a sequence selected from the group consisting of SEQ ID NOs: 10-73 and 75-193.

14-20. (canceled)

21. A method for isolating a protein of interest comprising: (a) culturing a recombinant host cell expressing a recombinant polynucleotide encoding an enterokinase cleavable fusion protein including the following domains, arranged in the direction of amino-terminus to carboxy-terminus: a ligand recognition sequence, an enterokinase recognition sequence having the formula: Xaa₁-Xaa₂-Xaa₃-Xaa₄-Asp-Arg-Xaa₅ (SEQ ID NO:206), wherein (i) Xaa₁ is an optional amino acid, which if present, is Ala, Asp, Glu, Phe, Gly, Ile, Asn, Ser, or Val; Xaa₂ is an optional amino acid which, if present, is Ala, Asp, Glu, His, Ile, Leu, Met, Gln or Ser; Xaa₃ is an optional amino acid residue which, if present, is Asp, Glu, Phe, His, Ile, Met, Asn, Pro, Val, or Trp; Xaa₄ is Ala, Asp, Glu, or Thr; and Xaa₅ can be any amino acid; or Xaa₁-Xaa₂-Xaa₃-Xaa₄-Glu-Arg-Xaa₅ (SEQ ID NO:207), (2) wherein Xaa₁ is an optional amino acid residue which, if present, is Asp or Glu; Xaa₂ is an optional amino acid residue which, if present, is Val; Xaa₃ is an optional amino acid residue which, if present, is Tyr; Xaa₄ is Asp, Glu, or Ser; and Xaa₅ can be any amino acid residue, and a protein of interest, under conditions suitable for expression of said fusion protein; (b) contacting the expressed fusion protein with a binding ligand immobilized on a solid support under conditions suitable for formation of a binding complex between the binding ligand and the ligand recognition sequence; (c) contacting the binding complex with enterokinase; and (d) recovering the protein of interest.

22. The method of claim 21, further comprising: step (a1) after step (a), wherein said fusion protein is not secreted on expression, of lysing the host cells, and (a2) separating the cellular debris from the culture medium.

23. The method of claim 21, further comprising: step (a1) after step (a), wherein said fusion protein is secreted on expression, of collecting culture media containing the secreted fusion protein.

24. The method according to claim 21, wherein said fusion protein has the formula: Z₁-Xaa₁-Xaa₂-Xaa₃-Xaa₄-Asp-Arg-Xaa₅-Z₂ (SEQ ID NO:1), wherein
(i) Xaa₁ is an optional amino acid, which if present, is Ala, Asp, Glu, Phe, Gly, Ile, Asn, Ser, or Val; Xaa₂ is an optional amino acid which, if present, is Ala, Asp, Glu, His, Ile, Leu, Met, Gln or Ser; Xaa₃ is an optional amino acid residue which, if present, is Asp, Glu, Phe, His, Ile, Met, Asn, Pro, Val, or Trp; Xaa₄ is Ala, Asp, Glu, or Thr; and
Xaa₅ can be any amino acid; Z₁ is a polypeptide comprising the sequence His-Pro-Gln-Phe-Ser-Ser-Pro-Ser-Ala-Ser-Arg-Pro-Ser-Glu-Gly-Pro-Cys-His-Pro-Gln-Phe-Pro-Arg-Cys-Tyr-Ile-Glu-Asn-Leu-Asp-Glu-Phe-Ser-Gly-Leu-Thr-Asn-Ile (SEQ ID NO:84), and Xaa₅-Z₂ is a protein of interest.

25. The method according to claim 21, wherein said fusion protein has the formula: Z₁-Xaa₁-Xaa₂-Xaa₃-Xaa₄-Glu-Arg-Xaa₅-Z₂ (SEQ ID NO:2), wherein (i) Xaa₁ is an optional amino acid, which if present, is Asp or Glu; Xaa₂ is an optional amino acid which, if present, is Val; Xaa₃ is an optional amino acid residue which, if present, is Tyr; Xaa₄ is Asp, Glu, or Ser; and Xaa₅ can be any amino acid; and Z₁ is a polypeptide comprising the sequence His-Pro-Gln-Phe-Ser-Ser-Pro-Ser-Ala-Ser-Arg-Pro-Ser-Glu-Gly-Pro-Cys-His-Pro-Gln-Phe-Pro-Arg-Cys-Tyr-Ile-Glu-Asn-Leu-Asp-Glu-Phe-Ser-Gly-Leu-Thr-Asn-Ile (SEQ ID NO:84), and Xaa₅-Z₂ is a protein of interest.

26. The method according to claim 24, wherein Xaa₅ is Met, Thr, Ser, Ala, Asp, Leu, Phe, Asn, Trp, Ile, Gln, Glu, His, Val, Gly, or Tyr.

27. A method for isolating a genetic package of interest comprising the steps: (a) expressing in a genetic package a fusion protein comprising a protein of interest fused to an enterokinase cleavage sequence fused to a polypeptide expressed on the surface of said genetic package; (b) contacting the genetic package with a ligand for the protein of interest, which ligand is capable of being immobilized on a solid support, under conditions suitable for the formation of a binding complex between said ligand and said protein of interest; (c) immobilizing said ligand on a solid support, either before or after said contacting step (b), (d) contacting the immobilized binding complex formed in step (b) with enterokinase; and (e) recovering the genetic package of interest from said solid support.

28. The method of claim 27, wherein the ligand is biotinylated and the immobilization is by binding to immobilized streptavidin or avidin.

29. The method of claim 27, wherein the ligand is immobilized by binding to an immobilized antibody that binds said ligand.

30-35. (canceled)

36. A method for controlling the activity of a protein of interest comprising the steps: (a) expressing in a recombinant host the polypeptide of claim 7 (b) treating the polypeptide with enterokinase such that said protein of interest is separated from the polypeptide and thereby exhibits the activity of the protein of interest.

37-49. (canceled)


SEQUENCE LISTING (217 sequences)

SEQ ID NO: 1 - protein, 7 aa, Artificial Sequence
  synthetic enterokinase cleavage sequence
XXXXDRX

SEQ ID NO: 2 - protein, 7 aa, Artificial Sequence
  synthetic enterokinase cleavage sequence
XXXXERX

SEQ ID NO: 3 - protein, 7 aa, Artificial Sequence
  synthetic enterokinase cleavage sequence
DINDDRX

SEQ ID NO: 4 - protein, 7 aa, Artificial Sequence
  synthetic enterokinase cleavage sequence
GNYTDRX

SEQ ID NO: 5 - protein, 6 aa, Artificial Sequence
  streptavidin binding sequence
CHPQFC

SEQ ID NO: 6 - protein, 4 aa, Artificial Sequence
  streptavidin binding sequence
HPQF

SEQ ID NO: 7 - protein, 9 aa, Artificial Sequence
  streptavidin binding sequence
CHPQFCSWR

SEQ ID NO: 8 - protein, 6 aa, Artificial Sequence
  synthetic enterokinase cleavage sequence
DDDDKX

SEQ ID NO: 9 - protein, 86 aa, Artificial Sequence
  exogenous display polypeptide of a phage display library
AEWHPQFSSPSASRPSEGPCHPQFPRCYIENLDEFRPGGSGGXXXXXXXXXXXXXGAQSDGGGSTEHAEGGSADPSYIEGRIVGSA

SEQ ID NO: 10 - protein, 7 aa, Artificial Sequence
  synthetic enterokinase cleavage sequence
YEWQDRT

SEQ ID NO: 11 - protein, 7 aa, Artificial Sequence
  synthetic enterokinase cleavage sequence
NSIKDRV

SEQ ID NO: 12 - protein, 7 aa, Artificial Sequence
  synthetic enterokinase cleavage sequence
AKATERH

SEQ ID NO: 13 - protein, 7 aa, Artificial Sequence
  synthetic enterokinase cleavage sequence
LGKVDRT

SEQ ID NO: 14 - protein, 7 aa, Artificial Sequence
  synthetic enterokinase cleavage sequence
GGMADKF

SEQ ID NO: 15 - protein, 7 aa, Artificial Sequence
  synthetic enterokinase cleavage sequence
GHWLDKN

SEQ ID NO: 16 - protein, 7 aa, Artificial Sequence
  synthetic enterokinase cleavage sequence
NKAKDRM

SEQ ID NO: 17 - protein, 7 aa, Artificial Sequence
  synthetic enterokinase cleavage sequence
SENFDKN

SEQ ID NO: 18 - protein, 7 aa, Artificial Sequence
  synthetic enterokinase cleavage sequence
LDWEDRA

SEQ ID NO: 19 - protein, 7 aa, Artificial Sequence
  synthetic enterokinase cleavage sequence
STDAERM

SEQ ID NO: 20 - protein, 7 aa, Artificial Sequence
  synthetic enterokinase cleavage sequence
HTFSDRQ

SEQ ID NO: 21 - protein, 7 aa, Artificial Sequence
  synthetic enterokinase cleavage sequence
GSGGDRL

SEQ ID NO: 22 - protein, 7 aa, Artificial Sequence
  synthetic enterokinase cleavage sequence
GFYNDRM

SEQ ID NO: 23 - protein, 7 aa, Artificial Sequence
  synthetic enterokinase cleavage sequence
IMPQDKS

SEQ ID NO: 24 - protein, 7 aa, Artificial Sequence
  synthetic enterokinase cleavage sequence
GGVEDRS

SEQ ID NO: 25 - protein, 7 aa, Artificial Sequence
  synthetic enterokinase cleavage sequence
WQESDRA

SEQ ID NO: 26 - protein, 7 aa, Artificial Sequence
  synthetic enterokinase cleavage sequence
GSGGDRH

SEQ ID NO: 27 - protein, 7 aa, Artificial Sequence
  synthetic enterokinase cleavage sequence
GHIFDRS

SEQ ID NO: 28 - protein, 7 aa, Artificial Sequence
  synthetic enterokinase cleavage sequence
GSGGEKL

SEQ ID NO: 29 - protein, 7 aa, Artificial Sequence
  synthetic enterokinase cleavage sequence
SGGEDRM

SEQ ID NO: 30 - protein, 7 aa, Artificial Sequence
  synthetic enterokinase cleavage sequence
GSGGERT

SEQ ID NO: 31 - protein, 7 aa, Artificial Sequence
  synthetic enterokinase cleavage sequence
PDPQERQ

SEQ ID NO: 32 - protein, 7 aa, Artificial Sequence
  synthetic enterokinase cleavage sequence
YIMGDRT

SEQ ID NO: 33 - protein, 7 aa, Artificial Sequence
  synthetic enterokinase cleavage sequence
QNHSDRT

SEQ ID NO: 34 - protein, 7 aa, Artificial Sequence
  synthetic enterokinase cleavage sequence
IAHGERA

SEQ ID NO: 35 - protein, 7 aa, Artificial Sequence
  synthetic enterokinase cleavage sequence
HEMNDRH

SEQ ID NO: 36 - protein, 7 aa, Artificial Sequence
  synthetic enterokinase cleavage sequence
THNGEKM

SEQ ID NO: 37 - protein, 7 aa, Artificial Sequence
  synthetic enterokinase cleavage sequence
HDEAEKT

SEQ ID NO: 38 - protein, 7 aa, Artificial Sequence
  synthetic enterokinase cleavage sequence
GYWIDRS

SEQ ID NO: 39 - protein, 7 aa, Artificial Sequence
  synthetic enterokinase cleavage sequence
GSGGERL

SEQ ID NO: 40 - protein, 7 aa, Artificial Sequence
  synthetic enterokinase cleavage sequence
SGGSDRL

SEQ ID NO: 41 - protein, 7 aa, Artificial Sequence
  synthetic enterokinase cleavage sequence
AQYMDLM

SEQ ID NO: 42 - protein, 7 aa, Artificial Sequence
  synthetic enterokinase cleavage sequence
GSGGERN

SEQ ID NO: 43 - protein, 7 aa, Artificial Sequence
  synthetic enterokinase cleavage sequence
GSGGENG

SEQ ID NO: 44 - protein, 7 aa, Artificial Sequence
  synthetic enterokinase cleavage sequence
ENYEERT

SEQ ID NO: 45 - protein, 7 aa, Artificial Sequence
  synthetic enterokinase cleavage sequence
NIYGDRI

SEQ ID NO: 46 - protein, 7 aa, Artificial Sequence
  synthetic enterokinase cleavage sequence
GGFVDKQ

SEQ ID NO: 47 - protein, 7 aa, Artificial Sequence
  synthetic enterokinase cleavage sequence
GSGGEKV

SEQ ID NO: 48 - protein, 7 aa, Artificial Sequence
  synthetic enterokinase cleavage sequence
GKFEDRN

SEQ ID NO: 49 - protein, 7 aa, Artificial Sequence
  synthetic enterokinase cleavage sequence
PAHTDRD

SEQ ID NO: 50 - protein, 7 aa, Artificial Sequence
  synthetic enterokinase cleavage sequence
QQMHDRF

SEQ ID NO: 51 - protein, 7 aa, Artificial Sequence
  synthetic enterokinase cleavage sequence
DMGYDRG

SEQ ID NO: 52 - protein, 7 aa, Artificial Sequence
  synthetic enterokinase cleavage sequence
SGGDEKE

SEQ ID NO: 53 - protein, 7 aa, Artificial Sequence
  synthetic enterokinase cleavage sequence
IESADRT

SEQ ID NO: 54 - protein, 7 aa, Artificial Sequence
  synthetic enterokinase cleavage sequence
RNMDERA

SEQ ID NO: 55 - protein, 7 aa, Artificial Sequence
  synthetic enterokinase cleavage sequence
TVGMDKF

SEQ ID NO: 56 - protein, 7 aa, Artificial Sequence
  synthetic enterokinase cleavage sequence
GSGGDRF

SEQ ID NO: 57 - protein, 7 aa, Artificial Sequence
  synthetic enterokinase cleavage sequence
RHNYDRI

SEQ ID NO: 58 - protein, 7 aa, Artificial Sequence
  synthetic enterokinase cleavage sequence
VYHVDKM

SEQ ID NO: 59 - protein, 7 aa, Artificial Sequence
  synthetic enterokinase cleavage sequence
GSGGERN

SEQ ID NO: 60 - protein, 7 aa, Artificial Sequence
  synthetic enterokinase cleavage sequence
GGKYDRM

SEQ ID NO: 61 - protein, 7 aa, Artificial Sequence
  synthetic enterokinase cleavage sequence
GGNDDKM

SEQ ID NO: 62 - protein, 7 aa, Artificial Sequence
  synthetic enterokinase cleavage sequence
AAVEDRN

SEQ ID NO: 63 - protein, 7 aa, Artificial Sequence
  synthetic enterokinase cleavage sequence
PCKDERF

SEQ ID NO: 64 - protein, 7 aa, Artificial Sequence
  synthetic enterokinase cleavage sequence
GSELDRM

SEQ ID NO: 65 - protein, 7 aa, Artificial Sequence
  synthetic enterokinase cleavage sequence
FSEEDRM

SEQ ID NO: 66 - protein, 7 aa, Artificial Sequence
  synthetic enterokinase cleavage sequence
GSGGERF

SEQ ID NO: 67 - protein, 7 aa, Artificial Sequence
  synthetic enterokinase cleavage sequence
YQPTDRT

SEQ ID NO: 68 - protein, 7 aa, Artificial Sequence
  synthetic enterokinase cleavage sequence
SGGEDRM

SEQ ID NO: 69 - protein, 7 aa, Artificial Sequence
  synthetic enterokinase cleavage sequence
TEQMDRM

SEQ ID NO: 70 - protein, 7 aa, Artificial Sequence
  synthetic enterokinase cleavage sequence
QPFDDRD

SEQ ID NO: 71 - protein, 7 aa, Artificial Sequence
  synthetic enterokinase cleavage sequence
GSGGERT

SEQ ID NO: 72 - protein, 7 aa, Artificial Sequence
  synthetic enterokinase cleavage sequence
EGMTDRL

SEQ ID NO: 73 - protein, 7 aa, Artificial Sequence
  synthetic enterokinase cleavage sequence
EIPEDRM

SEQ ID NO: 74 - protein, 7 aa, Artificial Sequence
  natural enterokinase cleavage sequence
GDDDDKI

SEQ ID NO: 75 - protein, 7 aa, Artificial Sequence
  synthetic enterokinase cleavage sequence
GSGGERS

SEQ ID NO: 76 - protein, 7 aa, Artificial Sequence
  synthetic enterokinase cleavage sequence
HGYEERM

SEQ ID NO: 77 - protein, 7 aa, Artificial Sequence
  synthetic enterokinase cleavage sequence
KPMEERM

SEQ ID NO: 78 - protein, 7 aa, Artificial Sequence
  synthetic enterokinase cleavage sequence
SGGNDRM

SEQ ID NO: 79 - protein, 7 aa, Artificial Sequence
  synthetic enterokinase cleavage sequence
GGTDDRF

SEQ ID NO: 80 - protein, 7 aa, Artificial Sequence
  synthetic enterokinase cleavage sequence
DVYSERM

SEQ ID NO: 81 - protein, 7 aa, Artificial Sequence
  synthetic enterokinase cleavage sequence
DVYSERM

SEQ ID NO: 82 - protein, 7 aa, Artificial Sequence
  synthetic enterokinase cleavage sequence
GSGGDRN

SEQ ID NO: 83 - protein, 7 aa, Artificial Sequence
  synthetic enterokinase cleavage sequence
DVTADDR

SEQ ID NO: 84 - protein, 7 aa, Artificial Sequence
  synthetic enterokinase cleavage sequence
AEFADRF

SEQ ID NO: 85 - protein, 7 aa, Artificial Sequence
  synthetic enterokinase cleavage sequence
NNSDEKI

SEQ ID NO: 86 - protein, 7 aa, Artificial Sequence
  synthetic enterokinase cleavage sequence
PGGDDRW

SEQ ID NO: 87 - protein, 7 aa, Artificial Sequence
  synthetic enterokinase cleavage sequence
SGGEERV

SEQ ID NO: 88 - protein, 7 aa, Artificial Sequence
  synthetic enterokinase cleavage sequence
VWPDDRS

SEQ ID NO: 89 - protein, 7 aa, Artificial Sequence
  synthetic enterokinase cleavage sequence
HRQTDRM

SEQ ID NO: 90 - protein, 7 aa, Artificial Sequence
  synthetic enterokinase cleavage sequence
KEAEDRA

SEQ ID NO: 91 - protein, 7 aa, Artificial Sequence
  synthetic enterokinase cleavage sequence
VGDDERH

SEQ ID NO: 92 - protein, 7 aa, Artificial Sequence
  synthetic enterokinase cleavage sequence
NSMADRN

SEQ ID NO: 93 - protein, 7 aa, Artificial Sequence
  synthetic enterokinase cleavage sequence
TEFEDKW

SEQ ID NO: 94 - protein, 7 aa, Artificial Sequence
  synthetic enterokinase cleavage sequence
ESGGERD

SEQ ID NO: 95 - protein, 7 aa, Artificial Sequence
  synthetic enterokinase cleavage sequence
NNYWDRM

SEQ ID NO: 96 - protein, 7 aa, Artificial Sequence
  synthetic enterokinase cleavage sequence
FSEEDRM

SEQ ID NO: 97 - protein, 7 aa, Artificial Sequence
  synthetic enterokinase cleavage sequence
EMHEERM

SEQ ID NO: 98 - protein, 7 aa, Artificial Sequence
  synthetic enterokinase cleavage sequence
DQMEDRQ

SEQ ID NO: 99 - protein, 7 aa, Artificial Sequence
  synthetic enterokinase cleavage sequence
EWKMDRM

SEQ ID NO: 100 - protein, 7 aa, Artificial Sequence
  synthetic enterokinase cleavage sequence
SYTWDRS

SEQ ID NO: 101 - protein, 7 aa, Artificial Sequence
  synthetic enterokinase cleavage sequence
SFMLDRM

SEQ ID NO: 102 - protein, 7 aa, Artificial Sequence
  synthetic enterokinase cleavage sequence
TEVDDRH

SEQ ID NO: 103 - protein, 7 aa, Artificial Sequence
  synthetic enterokinase cleavage sequence
GDQEDRM

SEQ ID NO: 104 - protein, 7 aa, Artificial Sequence
  synthetic enterokinase cleavage sequence
HNIDDRI

SEQ ID NO: 105 - protein, 7 aa, Artificial Sequence
  synthetic enterokinase cleavage sequence
ASWEDRT

SEQ ID NO: 106 - protein, 7 aa, Artificial Sequence
  synthetic enterokinase cleavage sequence
GGEDDRS

SEQ ID NO: 107 - protein, 7 aa, Artificial Sequence
  synthetic enterokinase cleavage sequence
DIQDERN

SEQ ID NO: 108 - protein, 7 aa, Artificial Sequence
  synthetic enterokinase cleavage sequence
DTHADKS

SEQ ID NO: 109 - protein, 7 aa, Artificial Sequence
  synthetic enterokinase cleavage sequence
GSGGDRM

SEQ ID NO: 110 - protein, 7 aa, Artificial Sequence
  synthetic enterokinase cleavage sequence
GEIMDRS

SEQ ID NO: 111 - protein, 7 aa, Artificial Sequence
  synthetic enterokinase cleavage sequence
GSGGDKT

SEQ ID NO: 112 - protein, 7 aa, Artificial Sequence
  synthetic enterokinase cleavage sequence
GSGGDRA

SEQ ID NO: 113 - protein, 7 aa, Artificial Sequence
  synthetic enterokinase cleavage sequence
GDHLDRM

SEQ ID NO: 114 - protein, 7 aa, Artificial Sequence
  synthetic enterokinase cleavage sequence
GQQDDRQ

SEQ ID NO: 115 - protein, 7 aa, Artificial Sequence
  synthetic enterokinase cleavage sequence
ALAADRM

SEQ ID NO: 116 - protein, 7 aa, Artificial Sequence
  synthetic enterokinase cleavage sequence
VGFDDRT

SEQ ID NO: 117 - protein, 7 aa, Artificial Sequence
  synthetic enterokinase cleavage sequence
YAQDERT

SEQ ID NO: 118 - protein, 7 aa, Artificial Sequence
  synthetic enterokinase cleavage sequence
GGREERN

SEQ ID NO: 119 - protein, 7 aa, Artificial Sequence
  synthetic enterokinase cleavage sequence
GSGGDRM

SEQ ID NO: 120 - protein, 7 aa, Artificial Sequence
  synthetic enterokinase cleavage sequence
GSGGDRE

SEQ ID NO: 121 - protein, 7 aa, Artificial Sequence
  synthetic enterokinase cleavage sequence
IAYQDRM

SEQ ID NO: 122 - protein, 7 aa, Artificial Sequence
  synthetic enterokinase cleavage sequence
SGGEDRA

SEQ ID NO: 123 - protein, 7 aa, Artificial Sequence
  synthetic enterokinase cleavage sequence
LEHSDRV

SEQ ID NO: 124 - protein, 7 aa, Artificial Sequence
  synthetic enterokinase cleavage sequence
FKPDDRM

SEQ ID NO: 125 - protein, 7 aa, Artificial Sequence
  synthetic enterokinase cleavage sequence
VPMADRS

SEQ ID NO: 126 - protein, 7 aa, Artificial Sequence
  synthetic enterokinase cleavage sequence
GSGGERA

SEQ ID NO: 127 - protein, 7 aa, Artificial Sequence
  synthetic enterokinase cleavage sequence
NDNDERA

SEQ ID NO: 128 - protein, 7 aa, Artificial Sequence
  synthetic enterokinase cleavage sequence
GNYTDRM

SEQ ID NO: 129 - protein, 7 aa, Artificial Sequence
  synthetic enterokinase cleavage sequence
GSGGERV

SEQ ID NO: 130 - protein, 7 aa, Artificial Sequence
  synthetic enterokinase cleavage sequence
DEVHDRT

SEQ ID NO: 131 - protein, 7 aa, Artificial Sequence
  synthetic enterokinase cleavage sequence
QHDGDKT

SEQ ID NO: 132 - protein, 7 aa, Artificial Sequence
  synthetic enterokinase cleavage sequence
TVRSEKG

SEQ ID NO: 133 - protein, 7 aa, Artificial Sequence
  synthetic enterokinase cleavage sequence
SGGTDRI

SEQ ID NO: 134 - protein, 7 aa, Artificial Sequence
  synthetic enterokinase cleavage sequence
VMEDDRA

SEQ ID NO: 135 - protein, 7 aa, Artificial Sequence
  synthetic enterokinase cleavage sequence
GSGGERM

SEQ ID NO: 136 - protein, 7 aa, Artificial Sequence
  synthetic enterokinase cleavage sequence
IEHDDRM

SEQ ID NO: 137 - protein, 7 aa, Artificial Sequence
  synthetic enterokinase cleavage sequence
FSEEDRM

SEQ ID NO: 138 - protein, 7 aa, Artificial Sequence
  synthetic enterokinase cleavage sequence
FSEEDRM

SEQ ID NO: 139 - protein, 7 aa, Artificial Sequence
  synthetic enterokinase cleavage sequence
DVYSERM

SEQ ID NO: 140 - protein, 7 aa, Artificial Sequence
  synthetic enterokinase cleavage sequence
DMFDDRM

SEQ ID NO: 141 - protein, 7 aa, Artificial Sequence
  synthetic enterokinase cleavage sequence
FSEEDRM

SEQ ID NO: 142 - protein, 7 aa, Artificial Sequence
  synthetic enterokinase cleavage sequence
EHLFDRM

SEQ ID NO: 143 - protein, 7 aa, Artificial Sequence
  synthetic enterokinase cleavage sequence
SWISDRV

SEQ ID NO: 144 - protein, 7 aa, Artificial Sequence
  synthetic enterokinase cleavage sequence
NDEDDRM

SEQ ID NO: 145 - protein, 7 aa, Artificial Sequence
  synthetic enterokinase cleavage sequence
SLDDDRT

SEQ ID NO: 146 - protein, 7 aa, Artificial Sequence
  synthetic enterokinase cleavage sequence
GSGGDRD

SEQ ID NO: 147 - protein, 7 aa, Artificial Sequence
  synthetic enterokinase cleavage sequence
PHIEDRM

SEQ ID NO: 148 - protein, 7 aa, Artificial Sequence
  synthetic enterokinase cleavage sequence
SGGDDRH

SEQ ID NO: 149 - protein, 7 aa, Artificial Sequence
  synthetic enterokinase cleavage sequence
EVFADRS

SEQ ID NO: 150 - protein, 7 aa, Artificial Sequence
  synthetic enterokinase cleavage sequence
GLAEDRT

SEQ ID NO: 151 - protein, 7 aa, Artificial Sequence
  synthetic enterokinase cleavage sequence
SGGDDRL

SEQ ID NO: 152 - protein, 7 aa, Artificial Sequence
  synthetic enterokinase cleavage sequence
SGGDDRM

SEQ ID NO: 153 - protein, 7 aa, Artificial Sequence
  synthetic enterokinase cleavage sequence
GLVSERG

SEQ ID NO: 154 - protein, 7 aa, Artificial Sequence
  synthetic enterokinase cleavage sequence
GGFEDKM

SEQ ID NO: 155 - protein, 7 aa, Artificial Sequence
  synthetic enterokinase cleavage sequence
SLDDDRT

SEQ ID NO: 156 - protein, 7 aa, Artificial Sequence
  synthetic enterokinase cleavage sequence
DVYSERM

SEQ ID NO: 157 - protein, 7 aa, Artificial Sequence
  synthetic enterokinase cleavage sequence
NMDWDRS

SEQ ID NO: 158 - protein, 7 aa, Artificial Sequence
  synthetic enterokinase cleavage sequence
SLDDDRT

SEQ ID NO: 159 - protein, 7 aa, Artificial Sequence
  synthetic enterokinase cleavage sequence
GSGGDRM

SEQ ID NO: 160 - protein, 7 aa, Artificial Sequence
  synthetic enterokinase cleavage sequence
FSEEDRM

SEQ ID NO: 161 - protein, 7 aa, Artificial Sequence
  synthetic enterokinase cleavage sequence
SLDDDRT

SEQ ID NO: 162 - protein, 7 aa, Artificial Sequence
  synthetic enterokinase cleavage sequence
VDMHDRM

SEQ ID NO: 163 - protein, 7 aa, Artificial Sequence
  synthetic enterokinase cleavage sequence
SGGDDRM

SEQ ID NO: 164 - protein, 7 aa, Artificial Sequence
  synthetic enterokinase cleavage sequence
NVRMDRS

SEQ ID NO: 165 - protein, 7 aa, Artificial Sequence
  synthetic enterokinase cleavage sequence
SHRDEKV

SEQ ID NO: 166 - protein, 7 aa, Artificial Sequence
  synthetic enterokinase cleavage sequence
LMNDDRA

SEQ ID NO: 167 - protein, 7 aa, Artificial Sequence
  synthetic enterokinase cleavage sequence
FVMNDKG

SEQ ID NO: 168 - protein, 7 aa, Artificial Sequence
  synthetic enterokinase cleavage sequence
VSDDDRA

SEQ ID NO: 169 - protein, 7 aa, Artificial Sequence
  synthetic enterokinase cleavage sequence
GHVDDRM

SEQ ID NO: 170 - protein, 7 aa, Artificial Sequence
  synthetic enterokinase cleavage sequence
HAIEERS

SEQ ID NO: 171 - protein, 7 aa, Artificial Sequence
  synthetic enterokinase cleavage sequence
DINDDRS

SEQ ID NO: 172 - protein, 7 aa, Artificial Sequence
  synthetic enterokinase cleavage sequence
GSGGERT

SEQ ID NO: 173 - protein, 7 aa, Artificial Sequence
  synthetic enterokinase cleavage sequence
AVIGDRS

SEQ ID NO: 174 - protein, 7 aa, Artificial Sequence
  synthetic enterokinase cleavage sequence
SGGEERG

SEQ ID NO: 175 - protein, 7 aa, Artificial Sequence
  synthetic enterokinase cleavage sequence
VEFYDRM

SEQ ID NO: 176 - protein, 7 aa, Artificial Sequence
  synthetic enterokinase cleavage sequence
GSGGERI

SEQ ID NO: 177 - protein, 7 aa, Artificial Sequence
  synthetic enterokinase cleavage sequence
SLDDDRT

SEQ ID NO: 178 - protein, 7 aa, Artificial Sequence
  synthetic enterokinase cleavage sequence
SGGQERS

SEQ ID NO: 179 - protein, 7 aa, Artificial Sequence
  synthetic enterokinase cleavage sequence
DINDDRS

SEQ ID NO: 180 - protein, 7 aa, Artificial Sequence
  synthetic enterokinase cleavage sequence
DHVWDRA

SEQ ID NO: 181 - protein, 7 aa, Artificial Sequence
  synthetic enterokinase cleavage sequence
GSGGDRI

SEQ ID NO: 182 - protein, 7 aa, Artificial Sequence
  synthetic enterokinase cleavage sequence
IEDEDRA

SEQ ID NO: 183 - protein, 7 aa, Artificial Sequence
  synthetic enterokinase cleavage sequence
MTFDERG

SEQ ID NO: 184 - protein, 7 aa, Artificial Sequence
  synthetic enterokinase cleavage sequence
GDWDDKN

SEQ ID NO: 185 - protein, 7 aa, Artificial Sequence
  synthetic enterokinase cleavage sequence
IAYQDRM

SEQ ID NO: 186 - protein, 7 aa, Artificial Sequence
  synthetic enterokinase cleavage sequence
GSGGDRI

SEQ ID NO: 187 - protein, 7 aa, Artificial Sequence
  synthetic enterokinase cleavage sequence
GFVQERM

SEQ ID NO: 188 - protein, 7 aa, Artificial Sequence
  synthetic enterokinase cleavage sequence
DINDDRS

SEQ ID NO: 189 - protein, 7 aa, Artificial Sequence
  synthetic enterokinase cleavage sequence
GWNDDRI

SEQ ID NO: 190 - protein, 7 aa, Artificial Sequence
  synthetic enterokinase cleavage sequence
GGFEDRL

SEQ ID NO: 191 - protein, 7 aa, Artificial Sequence
  synthetic enterokinase cleavage sequence
GSGGDRN

SEQ ID NO: 192 - protein, 7 aa, Artificial Sequence
  synthetic enterokinase cleavage sequence
AAVEDRN

SEQ ID NO: 193 - protein, 7 aa, Artificial Sequence
  synthetic enterokinase cleavage sequence
DYRLDRI

SEQ ID NO: 194 - protein, 7 aa, Artificial Sequence
  synthetic enterokinase cleavage sequence
GDDDDKI

SEQ ID NO: 195 - protein, 13 aa, Artificial Sequence
  synthetic enterokinase cleavage sequence
DRMYQLDKTGFMI

SEQ ID NO: 196 - protein, 13 aa, Artificial Sequence
  synthetic enterokinase cleavage sequence
AVLSNVMHSDDWT

SEQ ID NO: 197 - protein, 9 aa, Artificial Sequence
  natural enterokinase cleavage sequence
GDDDDKIYV

SEQ ID NO: 198 - protein, 9 aa, Artificial Sequence
  negative control in EK cleavage experiment
AVLSNVMFI

SEQ ID NO: 199 - protein, 9 aa, Artificial Sequence
  synthetic enterokinase cleavage sequence
GNYTDRMFI

SEQ ID NO: 200 - protein, 9 aa, Artificial Sequence
  synthetic enterokinase cleavage sequence
DINDDRSLF

SEQ ID NO: 201 - protein, 9 aa, Artificial Sequence
  synthetic enterokinase cleavage sequence
NKAKDRMFI

SEQ ID NO: 202 - protein, 9 aa, Artificial Sequence
  synthetic enterokinase cleavage sequence
GNYTDRRFI

SEQ ID NO: 203 - protein, 9 aa, Artificial Sequence
  commercial synthetic enterokinase cleavage substrate
GNYTDRYFI

SEQ ID NO: 204 - protein, 7 aa, Artificial Sequence
  synthetic enterokinase cleavage sequence
DINDDRX

SEQ ID NO: 205 - protein, 7 aa, Artificial Sequence
  synthetic enterokinase cleavage sequence
GNYTDRX

SEQ ID NO: 206 - protein, 7 aa, Artificial Sequence
  synthetic enterokinase cleavage sequence
XXXXDRX

SEQ ID NO: 207 - protein, 7 aa, Artificial Sequence
  synthetic enterokinase cleavage sequence
XXXXERX

SEQ ID NO: 208 - protein, 6 aa, Artificial Sequence
  synthetic enterokinase cleavage sequence
DINDDR

SEQ ID NO: 209 - protein, 6 aa, Artificial Sequence
  synthetic enterokinase cleavage sequence
GNYTDR

SEQ ID NO: 210 - protein, 7 aa, Artificial Sequence
  streptavidin binding sequence
WHPQFSS

SEQ ID NO: 211 - protein, 10 aa, Artificial Sequence
  steptavidin binding sequence
PCHPQFPRCY

SEQ ID NO: 212 - DNA, 1272 bp, Artificial Sequence
  Bacteriophage M13mp18
gtgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaagatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 213 - protein, 424 aa, Artificial Sequence
  Bacteriophage M13mp18
MKKLLFAIPLVVPFYSHSAETVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 214 - DNA, 957 bp, Artificial Sequence
  Bacteriophage M13mp18
aaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaagatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 215 - protein, 319 aa, Artificial Sequence
  Bacteriophage M13mp18
KPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 216 - DNA, 450 bp, Artificial Sequence
  Bacteriophage M13mp18
gattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtct

SEQ ID NO: 217 - protein, 150 aa, Artificial Sequence
  Bacteriophage M13mp18
DFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES